SEQUENCE LISTING (163 sequences)

SEQ ID NO: 1 - protein, 63 aa, Plasmodium falciparum
VNNLDSTVNYMNSTGNNINNIVNNLDSTVNYMNSTGNNINNIVNNLDSTVNYMNSTGNNINNI

SEQ ID NO: 2 - protein, 27 aa, Plasmodium falciparum
TISSLSNKIVNYESKIEELEKELKEVK

SEQ ID NO: 3 - protein, 27 aa, Plasmodium falciparum
NIKTMNTQISTLKNDVHLLNEQIDKLN

SEQ ID NO: 4 - protein, 27 aa, Plasmodium falciparum
EIEKLNKQLTKCNKQIDELNEEVEKLN

SEQ ID NO: 5 - protein, 25 aa, Plasmodium falciparum
IIDIKKHLEKLKIEIKEKKEDLENL

SEQ ID NO: 6 - protein, 25 aa, Plasmodium falciparum
INNLNEKLEETNKEYTNLQNNYTNE

SEQ ID NO: 7 - protein, 25 aa, Plasmodium falciparum
IDKLNNEKGTLNSKISELNVQIMDL

SEQ ID NO: 8 - protein, 46 aa, Plasmodium falciparum
IKTMNTQISTLKNDVHLLNEQIDKLNNEKGTLNSKISELNVQIMDL

SEQ ID NO: 9 - protein, 25 aa, Plasmodium falciparum
LLSKDKEIEEKNKKIKELNNDIKKL

SEQ ID NO: 10 - protein, 25 aa, Plasmodium falciparum
LNLVDQGKKKLKKDVEKQKKEIEKL

SEQ ID NO: 11 - protein, 49 aa, Plasmodium falciparum
ICSLTTEVMELNNKKNELIEENNKLNLVDQGKKKLKKDVEKQKKEIEKL

SEQ ID NO: 12 - protein, 77 aa, Plasmodium falciparum
VDKIEEHILDYDEEINKSRSNLFQLKNEICSLTTEVMELNNKKNELIEENNKLNLVDQGKKKLKKDVEKQKKEIEKL

SEQ ID NO: 13 - protein, 25 aa, Plasmodium falciparum
LDENEDNIKKMKSKIDDMEKEIKYR

SEQ ID NO: 14 - protein, 41 aa, Plasmodium falciparum
GMNNMNGDINNINGDINNMNGDINNMNGDINNMNGDINNMN

SEQ ID NO: 15 - protein, 27 aa, Plasmodium falciparum
LIKYMNERYQNMQQGYNNLTNYINQYE

SEQ ID NO: 16 - protein, 27 aa, Plasmodium falciparum
DINSTNNNLDNMLSEINSIQNNIHTYI

SEQ ID NO: 17 - protein, 22 aa, Plasmodium falciparum
EKKLDILKVNISNINNSLDKLK

SEQ ID NO: 18 - protein, 27 aa, Plasmodium falciparum
DVHNIKEDYNLLQQYLNYMKNEMEQLK

SEQ ID NO: 19 - protein, 25 aa, Plasmodium falciparum
DEKINDYLEEIKNEQNKIDKTIDDI

SEQ ID NO: 20 - protein, 25 aa, Plasmodium falciparum
MDVVINQLRDIDRQMLDLYKELDEK

SEQ ID NO: 21 - protein, 25 aa, Plasmodium falciparum
ISNIFKDIQNIKKQSQDIITNMNDM

SEQ ID NO: 22 - protein, 25 aa, Plasmodium falciparum
LEEIIKNLDILDEQIMTYHNSIDEL

SEQ ID NO: 23 - protein, 25 aa, Plasmodium falciparum
FQKVKEKAEIQKENIEKIKQEINTL

SEQ ID NO: 24 - protein, 54 aa, Plasmodium falciparum
EKKLDILKVNISNINNSLDKLKKYYEEALFQKVKEKAEIQKENIEKIKQEINTL

SEQ ID NO: 25 - protein, 34 aa, Plasmodium falciparum
DVTHLTNDVTHLTNDVTHLTNDVTHLTNDVTHLT

SEQ ID NO: 26 - protein, 27 aa, Plasmodium falciparum
NLNKVKININDLNNNIVDVNNSIHNIE

SEQ ID NO: 27 - protein, 27 aa, Plasmodium falciparum
KKRNVEEELHSLRKNYNIINEEIEEIT

SEQ ID NO: 28 - protein, 25 aa, Plasmodium falciparum
YIDIKKKISELQKDNESLKIQVDRL

SEQ ID NO: 29 - protein, 27 aa, Plasmodium falciparum
QMEGFQKQLDRLSDSLSKIQKALGEYL

SEQ ID NO: 30 - protein, 25 aa, Plasmodium falciparum
LKLNEGLENIKQELHIIDRELKNIL

SEQ ID NO: 31 - protein, 27 aa, Plasmodium falciparum
NINSVNNNINSVDNNINNVDNNINSVN

SEQ ID NO: 32 - protein, 27 aa, Plasmodium falciparum
NIIQIKNDIEQCQKSIKKIEDNLNTYE

SEQ ID NO: 33 - protein, 27 aa, Plasmodium falciparum
YIDDVDRDVENYDKGIANVDHHLNDVH

SEQ ID NO: 34 - protein, 27 aa, Plasmodium falciparum
NMNNMNNNMNNMNNNMNNNMNNMNNMN

SEQ ID NO: 35 - protein, 27 aa, Plasmodium falciparum
ARDDIQKDINKMESELINVSNEINRLD

SEQ ID NO: 36 - protein, 27 aa, Plasmodium falciparum
SSNNLSDQINILNNNIQHINSTFNNLR

SEQ ID NO: 37 - protein, 27 aa, Plasmodium falciparum
NNNVNNINMNNINSNVNNINNSMNNIN

SEQ ID NO: 38 - protein, 27 aa, Plasmodium falciparum
NITNINKNIENIKNDMSNLNNMNDSNQ

SEQ ID NO: 39 - protein, 36 aa, Plasmodium falciparum
NHDTRINDYNKRLTEYNKRLTEYNKRLTEYTKRLNE

SEQ ID NO: 40 - protein, 25 aa, Plasmodium falciparum
MEQMDMKMEKIDVNMDQMDVKMEQM

SEQ ID NO: 41 - protein, 30 aa, Plasmodium falciparum
NNVIRSKMYNIKKRISKINDELHELSNFFL

SEQ ID NO: 42 - protein, 25 aa, Plasmodium falciparum
LEEMKQKNKELINNLNDISDELKNC

SEQ ID NO: 43 - protein, 29 aa, Plasmodium falciparum
EKLNDMQKKLNDVNEKYKNIVECLNNYKT

SEQ ID NO: 44 - protein, 25 aa, Plasmodium falciparum
LKEKDKKINDLMKDMEKKKEEINKL

SEQ ID NO: 45 - protein, 40 aa, Plasmodium falciparum
NEIKELNNTLNKYKEEMNNYKEEIIVINEKYKLLEIELCK

SEQ ID NO: 46 - protein, 25 aa, Plasmodium falciparum
MNIMENDMNIMENDMNIIKNDMNIM

SEQ ID NO: 47 - protein, 25 aa, Plasmodium falciparum
MNIMEKDMNIIKNDMNIIKNNMNII

SEQ ID NO: 48 - protein, 29 aa, Plasmodium falciparum
EEIYKLNNDIDMLSNNCKKLKESIMMMEK

SEQ ID NO: 49 - protein, 29 aa, Plasmodium falciparum
KEIQMLKNQILSLEESIKSLNEFINNLKN

SEQ ID NO: 50 - protein, 31 aa, Plasmodium falciparum
KNVIELKEYLEDLKKRMFDMQKRLNDIIITK

SEQ ID NO: 51 - protein, 25 aa, Plasmodium falciparum
IETLENNLKIGKEKINKFDNEIQKL

SEQ ID NO: 52 - protein, 25 aa, Plasmodium falciparum
INNMKEQIEDVNHKIASINKEKEEL

SEQ ID NO: 53 - protein, 25 aa, Plasmodium falciparum
LKELEKKNDILSNDKNQLQKNLTQL

SEQ ID NO: 54 - protein, 25 aa, Plasmodium falciparum
LSEGNKELEKLEKNIKELEETNNTL

SEQ ID NO: 55 - protein, 25 aa, Plasmodium falciparum
LNHLTNNLSHHNNHMNHHTSNLNHI

SEQ ID NO: 56 - protein, 38 aa, Plasmodium falciparum
STDINSLNDEVKKLKEELNKIRNEYDDFKNKLELLYQK

SEQ ID NO: 57 - protein, 25 aa, Plasmodium falciparum
INNMDEKINNVDEQNNNMDEKINNV

SEQ ID NO: 58 - protein, 34 aa, Plasmodium falciparum
DNNVNNMDNNVNNVDNNVNNVDNNLNNVDNNVNN

SEQ ID NO: 59 - protein, 29 aa, Plasmodium falciparum
TLIDSFNLNLSYLRESINNKKKHINKIND

SEQ ID NO: 60 - protein, 25 aa, Plasmodium falciparum
LLNLQNDIQKKNKTLEELGEEIQKY

SEQ ID NO: 61 - protein, 33 aa, Plasmodium falciparum
MSQEKISEIIKDISALKTSCEKLNSQLDELITQ

SEQ ID NO: 62 - protein, 25 aa, Plasmodium falciparum
FNDVTSNVGNMNNNLTNFSNPYNSV

SEQ ID NO: 63 - protein, 37 aa, Plasmodium falciparum
IPLNQKVLEISKKLNNMNNNINEYKNYLSNFIHMLKE

SEQ ID NO: 64 - protein, 35 aa, Plasmodium falciparum
NNINNINNNINNINNNINNINNNINNINNNVNNYY

SEQ ID NO: 65 - protein, 25 aa, Plasmodium falciparum
MNNMNNNMNNLNNSINNNNMNNINV

SEQ ID NO: 66 - protein, 25 aa, Plasmodium falciparum
LKKKDEEIKHLNESIYILQQNMNNL

SEQ ID NO: 67 - protein, 25 aa, Plasmodium falciparum
FDAYNEKLGSISQSIDEIKKKIDNL

SEQ ID NO: 68 - protein, 33 aa, Plasmodium falciparum
DSMNNHKDDMNNYNDNINNYVESMNNYDDIMNK

SEQ ID NO: 69 - protein, 30 aa, Plasmodium falciparum
NNFVNNKMNNMNNMKNNMNNMNNIMNNIMN

SEQ ID NO: 70 - protein, 25 aa, Plasmodium falciparum
IDVIKNKHNKLNEKINSLEEKLIEM

SEQ ID NO: 71 - protein, 31 aa, Plasmodium falciparum
SNKTFEKLNEKLNDIRNDVTNYKNELEEFKN

SEQ ID NO: 72 - protein, 39 aa, Plasmodium falciparum
NSLDYYKKVIIKLKNNINNMEEYTNNITNDINVLKAHID

SEQ ID NO: 73 - protein, 25 aa, Plasmodium falciparum
MDTVYKNIINMSNNMTQMYNSMNNM

SEQ ID NO: 74 - protein, 25 aa, Plasmodium falciparum
VNKMNEEVNKMNEEVNKMNEEVNKM

SEQ ID NO: 75 - protein, 25 aa, Plasmodium falciparum
VNKMNKEVNKMDEEVNKMNKEVNKM

SEQ ID NO: 76 - protein, 25 aa, Plasmodium falciparum
LKSVDNYLEKINNKIKDLDDNINDR

SEQ ID NO: 77 - protein, 25 aa, Plasmodium falciparum
INNLETNINDYNKKIKEGDSQLNNI

SEQ ID NO: 78 - protein, 28 aa, Plasmodium falciparum
EKLKKYNNEISSLKKELDILNEKMGKCT

SEQ ID NO: 79 - protein, 25 aa, Plasmodium falciparum
FDNVNENFESINEPYDSINEPYDNI

SEQ ID NO: 80 - protein, 25 aa, Plasmodium falciparum
FNRLNKELNELRERYDHIIKHKKQM

SEQ ID NO: 81 - protein, 25 aa, Plasmodium falciparum
LQDLKKKKNLLQEEITNYKEEIKTL

SEQ ID NO: 82 - protein, 25 aa, Plasmodium falciparum
LIILDNKEKQNQQNINHLKEEINSM

SEQ ID NO: 83 - protein, 25 aa, Plasmodium falciparum
INEEENSLNEMKEDINEYVEMENKL

SEQ ID NO: 84 - protein, 25 aa, Plasmodium falciparum
MNKLKDEEKKIQEEEKKLNEELENI

SEQ ID NO: 85 - protein, 25 aa, Plasmodium falciparum
LSGLQNSLSGLKTPLSGLQNSLSGL

SEQ ID NO: 86 - protein, 37 aa, Plasmodium falciparum
EEIKEEIKEVKEEIKEVKEEIKEVKEEIKEVKEEIKE

SEQ ID NO: 87 - protein, 41 aa, Plasmodium falciparum
IEINMLTNNLLREMMKIKNKLQKLSNLLNALRSNIEKILKN

SEQ ID NO: 88 - protein, 36 aa, Plasmodium falciparum
KGLEEANEKLQIVREKVQSLKAKLSELISQYDHAIY

SEQ ID NO: 89 - protein, 25 aa, Plasmodium falciparum
VETLEKKLKEIYNDEQNYKNSLQNI

SEQ ID NO: 90 - protein, 25 aa, Plasmodium falciparum
IDKINENINRIKNNIKKLNDDINEL

SEQ ID NO: 91 - protein, 25 aa, Plasmodium falciparum
LQKLEEDKINIYKNLNNLNQELNQL

SEQ ID NO: 92 - protein, 25 aa, Plasmodium falciparum
LEEKTKQYNDLQNNMKTIKEQNEHL

SEQ ID NO: 93 - protein, 40 aa, Plasmodium falciparum
RLINNIEEIYNSNCEQIQNVRDEFAELKNDLNKIMNLINI

SEQ ID NO: 94 - protein, 25 aa, Plasmodium falciparum
IKEINKSLTEVKNELTELQKNQEEA

SEQ ID NO: 95 - protein, 31 aa, Plasmodium falciparum
SSISSSLTNISSSLTNISSSLTNISSSLSNS

SEQ ID NO: 96 - protein, 30 aa, Plasmodium falciparum
MCELNVMENNMNNIHSNNNNISTHMDDVIE

SEQ ID NO: 97 - protein, 25 aa, Plasmodium falciparum
LKEYSSKLQEREKKLKEKKNELQKV

SEQ ID NO: 98 - protein, 25 aa, Plasmodium falciparum
IDNINKNINCINNDVDNINSNINNI

SEQ ID NO: 99 - protein, 34 aa, Plasmodium falciparum
KIQIEEIKKETNQINKDIDHIEMNIINLKKKIEF

SEQ ID NO: 100 - protein, 25 aa, Plasmodium falciparum
IEIEREKINQLQEEIKKLQNEKNDL

SEQ ID NO: 101 - protein, 30 aa, Plasmodium falciparum
TYTLSKLNNQINELTKKINILRGNLDKARK

SEQ ID NO: 102 - protein, 35 aa, Plasmodium falciparum
KNDINVQLDDINVQLDDINVQLDDINIQLDEINLN

SEQ ID NO: 103 - protein, 25 aa, Plasmodium falciparum
KEEINEKLQLLNNDLDKKNEELNIL

SEQ ID NO: 104 - protein, 25 aa, Plasmodium falciparum
IKNLNNEINTLNDMLKDSEEEIRML

SEQ ID NO: 105 - protein, 25 aa, Plasmodium falciparum
LNHIINELEIKREEINQMNNKLNEL

SEQ ID NO: 106 - protein, 25 aa, Plasmodium falciparum
VKILKKRLNSISNDLEKRTEEIEHL

SEQ ID NO: 107 - protein, 25 aa, Plasmodium falciparum
INVLNDEITKLKNEINTYKNDLKNI

SEQ ID NO: 108 - protein, 25 aa, Plasmodium falciparum
INKLNQQINYLQDDINSKSDNIISL

SEQ ID NO: 109 - protein, 30 aa, Plasmodium falciparum
NFFLEQMENDMSSTYDKMNRINMDLSKLKR

SEQ ID NO: 110 - protein, 29 aa, Plasmodium falciparum
EKLYILEKSINKLKKLLNDINNKYQTIKK

SEQ ID NO: 111 - protein, 25 aa, Plasmodium falciparum
LNQVLEKYEELKKNINEYSKEENKL

SEQ ID NO: 112 - protein, 33 aa, Plasmodium falciparum
TSFSKYVRQLEQYFDNFDQDFLSLRQKISDILQ

SEQ ID NO: 113 - protein, 30 aa, Plasmodium falciparum
EKLVKHLDVIDKLIENIYDNINNLNEYINK

SEQ ID NO: 114 - protein, 25 aa, Plasmodium falciparum
INSHNNNMNNNNDNMNNMNNNNNNI

SEQ ID NO: 115 - protein, 25 aa, Plasmodium falciparum
INEIEKKIEDIEKNINITKENLKEL

SEQ ID NO: 116 - protein, 25 aa, Plasmodium falciparum
LKELENKITELQSSFSSYENEMKHV

SEQ ID NO: 117 - protein, 25 aa, Plasmodium falciparum
IDDIDRNAEQINKNYEHVNKNYEHV

SEQ ID NO: 118 - protein, 25 aa, Plasmodium falciparum
IDRQRDKINELEKKLEELRSSSEEL

SEQ ID NO: 119 - protein, 37 aa, Plasmodium falciparum
TIVQNSYNSFSDINKNINDIDKEMKTLIPMLDELLNE

SEQ ID NO: 120 - protein, 25 aa, Plasmodium falciparum
ISELEQEFNNNNQKLDNILQDINAM

SEQ ID NO: 121 - protein, 32 aa, Plasmodium falciparum
MEIKTIVQNSYNSFSDINKNINDIDKEMKTLI

SEQ ID NO: 122 - protein, 25 aa, Plasmodium falciparum
MLSLQNNIDKLKKSNNNLNEDLRKK

SEQ ID NO: 123 - protein, 25 aa, Plasmodium falciparum
MNLLREILKLMTDNIDTLKDKINEI

SEQ ID NO: 124 - protein, 29 aa, Plasmodium falciparum
GGLKNSNHNLNNIEMKYNTLNNNMNSINK

SEQ ID NO: 125 - protein, 33 aa, Plasmodium falciparum
PYLRRAKHNLNNLQGGINNLYSSVNVVYDNLFN

SEQ ID NO: 126 - protein, 25 aa, Plasmodium falciparum
LNSLERTVASLKNNEKQLHNNIQKI

SEQ ID NO: 127 - protein, 32 aa, Plasmodium falciparum
SLLDTLEKSVKGIDENIEKYNKELNVIKQKIE

SEQ ID NO: 128 - protein, 31 aa, Plasmodium falciparum
NNEMDETINKLKKDINKLNEKIEKYDNFMKM

SEQ ID NO: 129 - protein, 25 aa, Plasmodium falciparum
IDELENKIEKLKNELSKNSHNNNNI

SEQ ID NO: 130 - protein, 25 aa, Plasmodium falciparum
INNIDDHINNIDDHINNIDDHINNI

SEQ ID NO: 131 - protein, 25 aa, Plasmodium falciparum
INNIDDNKNNIDDHINNIDDHINNI

SEQ ID NO: 132 - protein, 25 aa, Plasmodium falciparum
LKSLNEKIKNYDSIIEEQKNQLENL

SEQ ID NO: 133 - protein, 140 aa, Plasmodium falciparum
MIKNPYSKIKNEKSFIDLNFIKGYSNFIKKKCVPYDKKKYIFCNKNRLISNVGNKINKKESEEQYDSDDDNDKLVLNDDEDDEKKQVHINNKTEATNITNINKNIENIKNDMSNLNNMNDSNQKIKMKNKGKYDSDVIIM

SEQ ID NO: 134 - protein, 42 aa, Plasmodium falciparum
KLEEMKQKNKELINNLNDISDELKNCIEQVNSVSRNMANVEK

SEQ ID NO: 135 - protein, 48 aa, Plasmodium falciparum
KNMDTVYKNIINMSNNMTQMYNSMNNMSHNIINASHDMMDASGNINSH

SEQ ID NO: 136 - protein, 47 aa, Plasmodium falciparum
EKMNMKMEQMDMKMEKIDVNMDQMDVKMEQMDVKMEQMDVKMKRMNK

SEQ ID NO: 137 - protein, 55 aa, Plasmodium falciparum
KNKLNKKWEQINDHINNLETNINDYNKKIKEGDSQLNNIQLQCENIEQKINKIKE

SEQ ID NO: 138 - protein, 54 aa, Plasmodium falciparum
NEMNKEVNKMNEEVNKMNEEVNKMNEEVNKMNKEVNKMDEEVNKMNKEVNKMNK

SEQ ID NO: 139 - protein, 57 aa, Plasmodium falciparum
EIINEIEKKIEDIEKNINITKENLKELENKITELQSSFSSYENEMKHVVKKIEDLEK

SEQ ID NO: 140 - protein, 70 aa, Plasmodium falciparum
QNKMENDMNIIKNDMNIMENDMNIMENDMNIIKNDMNIMEKDMNIIKNDMNIIKNNMNIIKNEMNIIKNV

SEQ ID NO: 141 - protein, 43 aa, Plasmodium falciparum
DFLDVIYYKLNIKEINKSLTEVKNELTELQKNQEEAKNILAFK

SEQ ID NO: 142 - protein, 41 aa, Plasmodium falciparum
ASIDNINKNINCINNDVDNINSNINNINDNIHKINSNVYGN

SEQ ID NO: 143 - protein, 41 aa, Plasmodium falciparum
NFIKELELQIKNLNNEINTLNDMLKDSEEEIRMLNHTLEEK

SEQ ID NO: 144 - protein, 40 aa, Plasmodium falciparum
KYKIEINVLNDEITKLKNEINTYKNDLKNINATLDFYKST

SEQ ID NO: 145 - protein, 40 aa, Plasmodium falciparum
NVLEYAELIIDRQRDKINELEKKLEELRSSSEELQKNVIK

SEQ ID NO: 146 - protein, 39 aa, Plasmodium falciparum
GIFIYNMNLLREILKLMTDNIDTLKDKINEIKCSYAFLK

SEQ ID NO: 147 - protein, 38 aa, Plasmodium falciparum
TKKLNKELSEGNKELEKLEKNIKELEETNNTLENDIKV

SEQ ID NO: 148 - protein, 38 aa, Plasmodium falciparum
NFVNNYINENILNLKSVDNYLEKINNKIKDLDDNINDR

SEQ ID NO: 149 - protein, 36 aa, Plasmodium falciparum
PDFDAYNEKLGSISQSIDEIKKKIDNLQKEIKVANK

SEQ ID NO: 150 - protein, 35 aa, Plasmodium falciparum
QLEEKTKQYNDLQNNMKTIKEQNEHLKNKFQSMGK

SEQ ID NO: 151 - protein, 31 aa, Plasmodium falciparum
ENINNMDEKINNVDEQNNNMDEKINNVDEKK

SEQ ID NO: 152 - protein, 30 aa, Plasmodium falciparum
EKGLKSLNEKIKNYDSIIEEQKNQLENLKM

SEQ ID NO: 153 - protein, 35 aa, Plasmodium falciparum
TISELEQEFNNNNQKLDNILQDINAMNLNINILQT

SEQ ID NO: 154 - protein, 83 aa, Plasmodium falciparum
  misc_feature (28)..(28): Xaa stands for PEG
KKRNVEEELHSLRKNYNIINEEIEEITXTISSLSNKIVNYESKIEELEKELKEVKXNITNINKNIENIKNDMSNLNNMNDSNQ

SEQ ID NO: 155 - protein, 105 aa, Plasmodium falciparum
  misc_feature (22)..(22): Xaa stands for PEG
VNNLDSTVNYMNSTGNNINNIXKKRNVEEELHSLRKNYNIINEEIEEITXTISSLSNKIVNYESKIEELEKELKEVKXNITNINKNIENIKNDMSNLNNMNDSNQ

SEQ ID NO: 156 - protein, 58 aa, Plasmodium falciparum
  misc_feature (29)..(29): Xaa stands for PEG
EKLKKYNNEISSLKKELDILNEKMGKCTXGGLKNSNHNLNNIEMKYNTLNNNMNSINK

SEQ ID NO: 157 - protein, 84 aa, Plasmodium falciparum
  misc_feature (26)..(26): Xaa stands for PEG
LDENEDNIKKMKSKIDDMEKEIKYRXEKLKKYNNEISSLKKELDILNEKMGKCTXGGLKNSNHNLNNIEMKYNTLNNNMNSINK

SEQ ID NO: 158 - protein, 95 aa, Plasmodium falciparum
  misc_feature (39)..(39): Xaa stands for PEG
TKKLNKELSEGNKELEKLEKNIKELEETNNTLENDIKVXEKLKKYNNEISSLKKELDILNEKMGKCTXKKRNVEEELHSLRKNYNIINEEIEEIT

SEQ ID NO: 159 - protein, 55 aa, Plasmodium falciparum
KDKMHQEMEKFKKDRKNLQLNLKNTRKNHEFLKNKMQNLVLTMKKSTADDKRFQY

SEQ ID NO: 160 - protein, 44 aa, Plasmodium falciparum
EIIDKDIIYMKSRINIMRENADKNNQKYDKIVSQKDKMHQEMEK

SEQ ID NO: 161 - protein, 54 aa, Plasmodium falciparum
IKELEETNNTLENDIKVEMNKGNLYKSRLALLKKNKVRISKAQEIIDKDIIYMK

SEQ ID NO: 162 - protein, 86 aa, Plasmodium falciparum
SIQRIKHLEGLTKKLNKELSEGNKELEKLEKNIKELEETNNTLENDIKVEMNKGNLYKSRLALLKKNKVRISKAQEIIDKDIIYMK

SEQ ID NO: 163 - protein, 60 aa, Plasmodium falciparum
MRHKISENEIINKIDSINLKEVKDASACMNNYTNFISIKLKKNREGIIHSIQRIKHLEGL

(19) United States
(12) Patent Application Publication
(10) Pub. No.: US 2009/0053265 A1
(43) Pub. Date: Feb. 26, 2009

(54) Antigenic peptides and their use

(76) Inventors: Giampietro Corradin, Epalinges (CH); Andrey Kajava, Montpellier Cedex (FR); Pierre Druilhe, Paris (FR); Ali Jafarshad, Paris (FR)

Correspondence Address: HARNESS, DICKEY & PIERCE, P.L.C., P.O. BOX 8910, RESTON, VA, 20195, US

(21) Appl. No.: 11/990,602

(22) PCT Filed: Aug. 16, 2006
(86) PCT No.: PCT/IB2006/002232

§ 371 Date: Feb. 19, 2008

(30) Foreign Application Priority Data
Aug. 19, 2005 (EP) 05018052.0

(51) Int. Cl. A61K 39/015; C07K 14/445; C07K 16/20; C07H 21/04; C12N 15/63; C12N 5/06; G01N 33/569; A61P 33/02

(52) U.S. Cl. 424/272.1; 530/324; 530/387.9; 536/23.1; 435/320.1; 435/325; 435/7.1

(57) ABSTRACT

This invention relates generally to the field of pathogen peptidic antigens and their use, for example, for the preparation of a vaccine against said pathogen. More specifically, the present invention relates to an antigenic peptide deriving from Plasmodium species sequences, and includes antibodies and methods of producing and using same.

### FIELD OF THE INVENTION

This invention relates generally to the field of pathogen peptidic antigens and their use, for example, for the preparation of a vaccine against said pathogen. More specifically, the present invention relates to an antigenic peptide deriving from plasmodium species sequences, and includes antibodies and methods of producing and using same.

### BACKGROUND OF THE INVENTION

Human Plasmodium falciparum (Pf) is a public health problem. Today approximately 40% of the world's population mostly those living in the poorest countries are at risk of malaria. Malaria causes more than 300 million acute illnesses and at least one million deaths annually. 90% of deaths due to malaria occur in sub-Saharan African countries mostly among young children and pregnant women (http://rbm.who.int).

Unfortunately, vaccine discovery is still a very empirical process given that protective antigens do not bear any structural, physicochemical or sequence related characteristics that would allow their identification. The only constraint for antigens whose protective capacity is antibody mediated is their accessibility to antibody molecules. Initially, vaccine development was performed with intact microorganisms or proteins, which were easily obtainable through classical purification methods. Introduction of molecular biology techniques generally improved the antigen isolation step, but difficulty in producing highly purified recombinant proteins in milligram amounts still remains; this renders this approach very costly and not adaptable to high throughput screening methods. Furthermore, proper protein folding still represents an additional problem in numerous cases. Thus, overcoming the bottlenecks of manufacturing and identification of proteins as targets of protection represents a grand challenge to the scientific community.

In the past 20 years huge efforts have been made to develop a malaria vaccine with over 40 phase I and II trials being conducted (Ballou et al, 2004, Van de Perre and Dedet, 2004). Currently, there are approximately 70 different vaccine candidates under development with at least 10 in clinical trials. The antigens used are derived from various stages of the parasite life cycle (Genton and Corradin, 2002, Ballou et al, 2004), with various immunisation regimens (Dunachie and Hill, 2003) and combined with different adjuvants formulated in various delivery systems (Genton et al, 2002; Taylor-Robinson, 2003; Ballou et al, 2004).

Results obtained in clinical trials conducted in endemic situations were encouraging and showed some levels of protection (Bojang et al, 1998, Bojang et al, 2001, Gentonet et al, 2002) while other trials showed no protection (Nosten et al, 1996, Valero et al, 1996, Urdaneta et al, 1998, Graves and Gelband, 2004). To date, the most advanced malaria vaccine is RTS,S/AS02A. This vaccine comprises a large portion of the immunologically dominant NANP repeats of the circumsporozoite protein and a portion of the C-terminal region containing T cell epitopes, which is fused to hepatitis B surface antigen (Gordon et al, 1995), co-expressed in Saccharomyces cerevisiae and formulated with the strong adjuvant AS02A (Stoute et al, 1997). RTS,S/AS02A targets the pre-erythrocytic liver stage and protects against both clinical malaria and parasitaemia in naive (Stoute et al, 1997) and malaria exposed adult males (Bojang et al, 2001), although for a short period. In addition, better protection level against clinical malaria has been obtained in toddlers in a recently completed trial in Mozambique (Alonso et al, 2004). Protection was sustained for up to 6 months in children, which are the most affected by the disease.

The vaccine candidates that have been used to date have not been systematically selected for their ability to protect; most of the parasite molecules were empirically prioritised following their chronological order of discovery. This approach though successful for other pathogens, has not been able to provide an effective malaria vaccine.

For example, EP 0315 0185 B1 (in the name of Behringwerke aktiengesellschaft) described the isolation of a gene which codes for a histidine/alanine-rich protein by screening two different Plasmodium falciparum cDNA gene banks with antiserum against a 41 kDa protein and by cross-hybridisation with the insert DNA of a clone obtained therewith. The isolated protein protected Aotus monkeys from infection with Plasmodium falciparum. Unfortunately, this approach had not lead to the development of an efficient vaccine in humans probably due to the fact that these monkeys are not the appropriate models to mimic human immunological response to Plasmodium falciparum.

In an attempt to develop vaccines against another pathogen, Streptococcus pneumoniae, mapping studies using monoclonal antibodies raised against pneumococcal surface protein A (PspA) indicated that the major cross-reactive epitopes are found in the last 100 amino-acids of the coiled-coil domain which is an important structural and biologically abundant domain found in diverse group of proteins (Chen and Parry, 1986 and 1990).

Pursuant to this finding, International Patent Application WO00/37105 (in the name of Medimmune, Inc.) disclosed a process and a vaccine composition for preventing infection caused by Streptococcus pneumoniae that comprises polypeptides and fragments of polypeptides containing histidine triad residues or coiled-coil regions.

More recently, International Patent Application WO01/96368 (in the name of Cytovax Biotechnologies, Inc.) disclosed the use of a coiled-coil structural scaffold to generate structure-specific peptides, including synthetic peptides. These synthetic peptides are useful as vaccines or to stimulate antibody production or cell-mediated immunity to the naturally occurring pneumococcal surfaces proteins A and C.

Although there has been some progress in the treatment of malaria, the development of a safe and effective malaria vaccine remains an urgent unmet medical need for vast populations living in malaria-endemic region.

This object has been achieved by providing a new antigenic peptide deriving from Plasmodium species comprising at least one coiled coil region. This peptide has been prepared according to a method of producing an antigenic peptide for the preparation of a vaccine against a pathogen based on a new approach for identifying the presence of a sequence encoding at least one coiled-coil region in the genome of said pathogen.

### SUMMARY OF THE INVENTION

These and other objects as will be apparent from the foregoing have been achieved by providing a new antigenic peptide deriving from Plasmodium species comprising at least one coiled coil region, characterized in that said antigenic peptide is selected from the group comprising the amino acid sequences SEQ ID NO 1 to 132, SEQ ID NO 134 to SEQ ID NO 153 and SEQ ID NO 159 to SEQ ID NO 163, biologically active fragments thereof, molecular chimeras thereof, combinations thereof and/or variants thereof.

Another object of the invention is to provide an antigenic cocktail composition deriving from Plasmodium species.

This invention also contemplates the use of the antigenic peptide deriving from Plasmodium species or of the antigenic cocktail composition in the preparation of a vaccine composition useful to stimulate an immune response in a mammal and the use thereof in the manufacture of a medicament for the treatment and/or prevention of malaria

A further object of the present invention is to provide an antibody that recognizes the antigenic peptide of or the antigenic cocktail composition deriving from Plasmodium species.

The present invention also relates to a purified and isolated nucleic acid sequence comprising a nucleotidic sequence encoding the antigenic peptide deriving from Plasmodium species, an expression vector comprising at least one copy of said purified and isolated nucleic acid sequence, and a host cell comprising either the purified and isolated nucleic acid sequence or the expression vector.

The present invention further relates to a method of producing an antigenic peptide for the preparation of a vaccine composition against a pathogen.

A diagnostic tool for determining the presence of the antigenic peptide or antibodies directed against said antigenic peptide is also contemplated in the present invention

Other objects and advantages will become apparent to those skilled in the art from a review of the ensuing detailed description, which proceeds with reference to the following illustrative drawings, and the attendant claims.

### BRIEF DESCRIPTION OF THE FIGURES

FIG. 1 represents the immunofluorence data obtained on infected human erythrocytes using affinity purified human antibodies against SEQ ID NO 34 (P1577), SEQ ID NO 4 (P1574), SEQ ID NO 133 (MR198) and SEQ ID NO 1 (MR194).

### DETAILED DESCRIPTION OF THE INVENTION

The present invention relates to an antigenic peptide deriving from Plasmodium species comprising at least one coiled coil region from a Plasmodium species sequence.

The term “comprising” is generally used in the sense of including, that is to say permitting the presence of one or more features or components.

As used herein, the terms “protein”, “polypeptide”, “polypeptidic”, “peptide” and “peptidic” are used interchangeably herein to designate a series of amino acid residues connected to the other by peptide bonds between the alpha-amino and carboxy groups of adjacent residues.

The term “antigenic peptide” refers to a peptide that is recognized by an antibody. Usually, the immune response results in the production of antibodies recognizing the antigenic peptide or at least a part thereof.

In the present context, the term “deriving” refers to the fact that the antigenic peptide has been selected among the amino acid sequences present in the Plasmodium species pathogen sequence.

“Coiled coil region”, “coiled-coil domain” or “region” are usually made up of amino acid sequence having heptad (abcdefg) repeats with apolar residues at the a and b positions and typically polar residues elsewhere. The structure of these regions are stabilized by hydrophobic interactions between the a and d positions in the different strands (Hodges et al, 1981; Hodges, 1996).

“Plasmodium species” as used herein can be selected among the following plasmodium strains:

Plasmodium agamae, Plasmodium atheruri, Plasmodium azurophilum, Plasmodium berghei, Plasmodium brasilianum, Plasmodium cathemerium, Plasmodium chabaudi, Plasmodium chiricahuae, Plasmodium coatneyi, Plasmodium cuculus, Plasmodium cynomolgi, Plasmodium elongatum, Plasmodium fairchildi, Plasmodium falciparum, (such as Plasmodium falciparum (isolate 311), Plasmodium falciparum (isolate 7G8), Plasmodium falciparum (isolate CAMP/Malaysia), Plasmodium falciparum (isolate CDC/Honduras), Plasmodium falciparum (isolate DD2), Plasmodium falciparum (isolate FC27/Papua New Guinea), Plasmodium falciparum (isolate FcB1/Columbia), Plasmodium falciparum (isolate FCBR/Columbia), Plasmodium falciparum (isolate FCH-5), Plasmodium falciparum (isolate FCM17/Senegal), Plasmodium falciparum (isolate FCR-3/Gambia), Plasmodium falciparum (isolate fid3/india), Plasmodium falciparum (isolate hb3), Plasmodium falciparum (isolate IMR143), Plasmodium falciparum (isolate K1/Thailand), Plasmodium falciparum (isolate KF1916), Plasmodium falciparum (isolate LE5), Plasmodium falciparum (isolate mad20/papua new guinea), Plasmodium falciparum (isolate mad71/papua new guinea), Plasmodium falciparum (isolate NF54), Plasmodium falciparum (isolate NF7/Ghana), Plasmodium falciparum (isolate nig32/nigeria), Plasmodium falciparum (isolate PALO ALTO/UGANDA), Plasmodium falciparum (isolate RO-33/Ghana), Plasmodium falciparum (isolate T4/Thailand), Plasmodium falciparum (isolate TAK 9), Plasmodium falciparum (isolate thtn/thailand), Plasmodium falciparum (isolate V1), Plasmodium falciparum (isolate WELLCOME), Plasmodium falciparum 3D7), Plasmodium fieldi, Plasmodium floridense, Plasmodium fragile, Plasmodium gallinaceum, Plasmodium giganteum, Plasmodium gonderi, Plasmodium guanggong, Plasmodium heteronucleare, Plasmodium hylobati, Plasmodium inui, Plasmodium juxtanucleare, Plasmodium knowlesi, Plasmodium lophurae, Plasmodium malariae, Plasmodium cf. malariae, Plasmodium mexicanum, Plasmodium nucleophilum, Plasmodium ovale, (malaria parasite P. ovale), Plasmodium reichenowi, Plasmodium relictum, Plasmodium rouxi, Plasmodium simiovale, Plasmodium simium, Plasmodium vinckei, Plasmodium vivax (such as Plasmodium vivax (strain Belem) Plasmodium vivax (strain Salvador I)), Plasmodium yoelii, Plasmodium sp. unclassified such as Plasmodium sp. 909413, Plasmodium sp. 909414, Plasmodium sp. 909415, Plasmodium sp. 909416, Plasmodium sp. AP61, Plasmodium sp. AP62, Plasmodium sp. AP63, Plasmodium sp. AP64, Plasmodium sp. AP65, Plasmodium sp. AP66, Plasmodium sp. AP67, Plasmodium sp. AP68, Plasmodium sp. AP69, Plasmodium sp. AP70, Plasmodium sp. AP71, Plasmodium sp. AP72, Plasmodium sp. AP73, Plasmodium sp. AP74, Plasmodium sp. AP75, Plasmodium sp. AP76, Plasmodium sp. AP77, Plasmodium sp. AP78, Plasmodium sp. C1, Plasmodium sp. C2, Plasmodium sp. C3, Plasmodium sp. COLL1, Plasmodium sp. D1, Plasmodium sp. DAJ-2004, Plasmodium sp. E1, Plasmodium sp. E2, Plasmodium sp. ex Aplonis atrifusca, Plasmodium sp. ex Aplonis tabuensis, Plasmodium sp. ex Foulehaio carunculata, Plasmodium sp. ex Halcyon chloris, Plasmodium sp. ex Myzomela cardinalis, Plasmodium sp. ex Ptilinopus porphyraceus, Plasmodium sp. F1, Plasmodium sp. G1, Plasmodium sp. GRW11, Plasmodium sp. GRW2, Plasmodium sp. GRW4, Plasmodium sp. H1, Plasmodium sp. ORW1, Plasmodium sp. PA, Plasmodium sp. PARUS2, Plasmodium sp. PB, Plasmodium sp. pBT6, Plasmodium sp. pBT7, Plasmodium sp. pBT8, Plasmodium sp. pBT9, Plasmodium sp. PC, Plasmodium sp. pGRW9, Plasmodium sp. PH, Plasmodium sp. PI, Plasmodium sp. PU1, Plasmodium sp. SGS1, Plasmodium sp. strain KZ02, Plasmodium sp. strain LA09, Plasmodium sp. strain LA15, Plasmodium sp. strain OZ01A, Plasmodium sp. strain OZ04, Plasmodium sp. strain OZ08, Plasmodium sp. strain OZ14, Plasmodium sp. strain OZ25, Plasmodium sp. strain OZ35, Plasmodium sp. strain OZ35A, Plasmodium sp. strain OZ35B, Plasmodium sp. strain OZ36A, Plasmodium sp. strain OZ42, Plasmodium sp. strain OZ45, Plasmodium sp. strain OZ46, Plasmodium sp. strain PR01, Plasmodium sp. SYAT24, Plasmodium vivax-like sp).

Preferably, the antigenic peptide will derive from Plasmodium falciparum, Plasmodium vivax, Plasmodium ovale and Plasmodium malariae.

Usually, the antigenic peptide deriving from Plasmodium species comprising at least one coiled coil region will be selected from the group comprising the amino acid sequences SEQ ID NO 1 to 132, SEQ ID NO 134 to SEQ ID NO 153 and SEQ ID NO 159 to SEQ ID NO 163, biologically active fragments thereof, molecular chimeras thereof, combinations thereof and/or variants thereof.

Applicants have selected SEQ ID NO 1 to 132, SEQ ID NO 134 to SEQ ID NO 153 and SEQ ID NO 159 to SEQ ID NO 163, represented in table 1, according to the method of producing an antigenic peptide described infra. All these peptidic sequences contain at least one coiled coil region.

TABLE 1

Sanger_1 3.030 303 pos. 497-559 Pfa3D7|pfa1_chr1|PFA0170c|Annotation|Sanger (protein coding) hypotheti-
cal protein, cons

| | |
|---|---|
| VNNLDSTVNYMNSTGNNINNIVNNLDSTVNYMNSTGNNINNIVNNLDSTVNYMNSTGNNINNI | (SEQ ID N^{o}1) |

Sanger 3.250 325 pos. 437-473 Pfa3D7|pfa1_chr1|PFA0635c|Annotation|Sanger (protein coding) hypothetical
protein Location

| | |
|---|---|
| NHDTRINDYNKRLTEYNKRLTEYNKRLTEYTKRLNE | (SEQ ID N^{o}39) |

TIGR_1 3.320 332 pos. 140-166 Pfa3D7|pfa1_chr2|PFB0145c|Annotation|TIGR (protein coding) hypothetical
protein Location

| | |
|---|---|
| TISSLSNKIVNYESKIEELEKELKEVK | (SEQ ID N^{o}2) |

TIGR_2 3.090 309 pos. 249-273 Pfa3D7|pfa1_chr2|PFB0145c|Annotation|TIGR (protein coding) hypothetical
protein Location

| | |
|---|---|
| IIDIKKHLEKLKIEIKEKKEDLENL | (SEQ ID N^{o}5) |

TIGR_3 3.640 364 pos. 575-601 Pfa3D7|pfa1_chr2|PFB0145c|Annotation|TIGR (protein coding) hypothetical
protein Location

| | |
|---|---|
| NIKTMNTQISTLKNDVHLLNEQIDKLN | (SEQ ID N^{o}3) |

TIGR_4 2.930 293 pos. 537-561 Pfa3D7|pfa1_chr2|PFB0145c|Annotation|TIGR (protein coding) hypothetical
protein Location

| | |
|---|---|
| INNLNEKLEETNKEYTNLQNNYTNE | (SEQ ID N^{o}6) |

TIGR_6 2.710 271 pos. 597-621 Pfa3D7|pfa1_ch2|PFB0145c|Annotation|TIGR (protein coding) hypothetical
protein Location

| | |
|---|---|
| IDKLNNEKGTLNSKISELNVQIMDL | (SEQ ID N^{o}7) |

TIGR_7 3.000 300 pos. 999-1023 Pfa3D7|pfa1_chr2|PFB0145c|Annotation|TIGR (protein coding) hypothetical
protein Location

| | |
|---|---|
| LLSKDKEIEEKNKKIKELNNDIKKL | (SEQ ID N^{o}9) |

TIGR_8 2.800 280 pos. 1154-1178 Pfa3D7|pfa1_chr2|PFB0145c|Annotation|TIGR (protein coding) hypothetical
protein Location

| | |
|---|---|
| ICSLTTEVMELNNKKNELIEENNKL | (SEQ ID N^{o}11) |

TIGR_9 2.690 269 pos. 1126-1150 Pfa3D7|pfa1_chr2|PFB0145c|Annotation|TIGR (protein coding) hypothetical
protein Location

| | |
|---|---|
| VDKIEEHILDYDEEINKSRSNLFQLKNE | (SEQ ID N^{o}12) |

TIGR_10 4.050 405 pos. 1198-1224 Pfa3D7|pfa1_chr2|PFB0145c|Annotation|TIGR (protein coding) hypotheti-
cal protein Location

| | |
|---|---|
| EIEKLNKQLTKCNKQIDELNEEVEKLN | (SEQ ID N^{o}4) |

TIGR_11 2.670 267 pos. 1178-1202 Pfa3D7|pfa1_chr2|PFB0145c|Annotation|TIGR (protein coding) hypotheti-
cal protein Location

| | |
|---|---|
| LNLVDQGKKKLKKDVEKQKKEIEKL | (SEQ ID N^{o}10) |

TIGR_12 2.600 260 pos. 1252-1276 Pfa3D7|pfa1_chr2|PFB0145c|Annotation|TIGR (protein coding) hypotheti-
cal protein Location

| | |
|---|---|
| LDENEDNIKKMKSKIDDMEKEIKYR | (SEQ ID N^{o}13) |

TIGR 3.170 317 pos. 1603-1627 Pfa3D7|pfa1_chr2|PFB0315w|Annotation|TIGR (protein coding) 41 kDa antigen
Location = 281907.

| | |
|---|---|
| MEQMDMKMEKIDVNMDQMDVKMEQM | (SEQ ID N^{o}40) |

TIGR 3.020 302 pos. 616-645 Pfa3D7|pfa1_chr2|PFB0460c|Annotation|TIGR (protein coding) hypothetical
protein Location = co

| | |
|---|---|
| NNVIRSKMYNIKKRISKINDELHELSNFFL | (SEQ ID N^{o}41) |

TIGR_1 3.160 316 pos. 516-540 Pfa3D7|pfa1_chr2|PFB0765w|Annotation|TIGR (protein coding) hypothetical
protein Location

| | |
|---|---|
| LEEMKQKNKELINNLNDISDELKNC | (SEQ ID N^{o}42) |

TIGR_2 3.470 347 pos. 605-633 Pfa3D7|pfa1_chr2|PFB0765w|Annotation|TIGR (protein coding) hypothetical
protein Location

| | |
|---|---|
| EKLNDMQKKLNDVNEKYKNIVECLNNYKT | (SEQ ID N^{o}43) |

TIGR_3 3.370 337 pos. 1074-1098 Pfa3D7|pfa1_chr2|PFB0765w|Annotation|TIGR (protein coding) hypothetical
protein Location

| | |
|---|---|
| LKEKDKKINDLMKDMEKKKEEINKL | (SEQ ID N^{o}44) |

Sanger 3.340 334 pos. 798-837 Pfa3D7|pfa1_chr3|PFC0235w|Annotation|Sanger (protein coding) hypothetical
protein Locat

| | |
|---|---|
| NEIKELNNTLNKYKEEMNNYKEEIIVINEKYKLLEIELCK | (SEQ ID N^{o}45) |

Sanger_1 4.000 400 pos. 2215-2255 Pfa3D7|pfa1_chr3|PFC0245c|Annotation|Sanger (protein coding) hypo-
thetical protein Location

| | |
|---|---|
| GMNNMNGDINNINGDINNMNGDINNMNGDINNMNGDINNMN | (SEQ ID N^{o}14) |

Sanger_1 3.220 322 pos. 285-309 PFA3D7|pfa1_chr3|PFC0345w|Annotation|Sanger (protein coding) hypotheti-
cal protein Locat

| | |
|---|---|
| MNIMENDMNIMENDMNIIKNDMNIM | (SEQ ID N^{o}46) |

Sanger_2 3.140 314 pos. 306-330 Pfa3D7|pfa1_chr3|PFC0345w|Annotation|Sanger (protein coding) hypotheti-
cal protein Locat

| | |
|---|---|
| MNIMEKDMNIIKNDMNIIKNNMNII | (SEQ ID N^{o}47) |

Sanger_1 3.020 302 pos. 2208-2236 Pfa3D7|pfa1_chr3|PFC0760c|Annotation|Sanger (protein coding) hypo-
thetical protein Locat

| | |
|---|---|
| EEIYKLNNDIDMLSNNCKKLKESIMMMEK | (SEQ ID N^{o}48) |

Sanger_2 3.430 343 pos. 2401-2429 Pfa3D7|pfa1_chr3|PFC0760c|Annotation|Sanger (protein coding) hypo-
thetical protein Locat

| | |
|---|---|
| KEIQMLKNQILSLEESIKSLNEFINNLKN | (SEQ ID N^{o}49) |

Sanger 3.640 364 pos. 180-210 Pfa3D7|pfa1_chr3|PFC0810c|Annotation|Sanger(protein coding) hypothetical
protein Locatio

| | |
|---|---|
| KNVIELKEYLEDLKKRMFDMQKRLNDIIITK | (SEQ ID N^{o}50) |

Sanger_1 3.010 301 pos. 1256-1280 Pfa3D7|pfa1_chr3|PFC0960c|Annotation|Sanger (protein coding) hypo-
thetical protein Locat

| | |
|---|---|
| IETLENNLKIGKEKINKFDNEIQKL | (SEQ ID N^{o}51) |

Sanger_4 3.160 316 pos. 1707-1731 Pfa3D7|pfa1_chr3|PFC0960c|Annotation|Sanger (protein coding) hypo-
thetical protein Locat

| | |
|---|---|
| INNMKEQIEDVNHKIASINKEKEEL | (SEQ ID N^{o}52) |

Sanger_1 2.620 262 pos. 758-782 Pfa3D7|pfa1_chr4|PFD0110w|Annotation|Sanger (protein coding) reticulo-
cyte binding protein

| | |
|---|---|
| DEKINDYLEEIKNEQNKIDKTIDDI | (SEQ ID N^{o}19) |

Sanger_2 3.270 327 pos. 813-839 Pfa3D7|pfa1_chr4|PFD0110w|Annotation|Sanger (protein coding) reticulo-
cyte binding protein

| | |
|---|---|
| LIKYMNERYQNMQQGYNNLTNYINQYE | (SEQ ID N^{o}15) |

Sanger_3 2.670 267 pos. 1396-1420 Pfa3D7|pfa1_chr4|PFD0110w|Annotation|Sanger (protein coding)
reticulocyte binding protein

| | |
|---|---|
| MDVVINQLRDIDRQMLDLYKELDEK | (SEQ ID N^{o}20) |

Sanger_4 3.090 309 pos. 1814-1840 Pfa3D7|pfa1_chr4|PFD0110w|Annotation|Sanger (protein coding)
reticulocyte binding protein

| | |
|---|---|
| DINSTNNNLDNMLSEINSIQNNIHTYI | (SEQ ID N^{o}16) |

Sanger_5 3.180 318 pos. 1904-1925 Pfa3D7|pfa1_chr4|PFD0110w|Annotation|Sanger (protein coding)
reticulocyte binding protein

| | |
|---|---|
| EKKLDILKVNISNINNSLDKLK | (SEQ ID N^{o}17) |

Sanger_6 2.620 262 pos. 1933-1957 Pfa3D7|pfa1_chr4|PFD0110w|Annotation|Sanger (protein coding)
reticulocyte binding protein

| | |
|---|---|
| FQKVKEKAEIQKENIEKIKQEINTL | (SEQ ID N^{o}23) |

Sanger_7 3.250 325 pos. 2070-2096 Pfa3D7|pfa1_chr4|PFD0110w|Annotation|Sanger (protein coding)
reticulocyte binding protein

| | |
|---|---|
| DVHNIKEDYNLLQQYLNYMKNEMEQLK | (SEQ ID N^{o}18) |

Sanger_8 2.930 293 pos. 2208-2232 Pfa3D7|pfa1_chr4|PFD0110w|Annotation|Sanger (protein coding)
reticulocyte binding protein

| | |
|---|---|
| ISNIFKDIQNIKKQSQDIITNMNDM | (SEQ ID N^{o}21) |

Sanger_9 2.950 295 pos. 2359-2383 Pfa3D7|pfa1_chr4|PFD0110w|Annotation|Sanger (protein coding)
reticulocyte binding protein

| | |
|---|---|
| LEEIIKNLDILDEQIMTYHNSIDEL | (SEQ ID N^{o}22) |

Sanger 3.170 317 pos. 35-59 Pfa3D7|pfa1_chr4|PFD0330w|Annotation|Sanger (protein coding) hypothetical
protein Location

| | |
|---|---|
| LKELEKKNDILSNDKNQLQKNLTQL | (SEQ ID N^{o}53) |

Sanger 3.490 349 pos. 68-92 Pfa3D7|pfa1_chr4|PFD0520c|Annotation|Sanger (protein coding) hypothetical
protein Location

| | |
|---|---|
| LSEGNKELEKLEKNIKELEETNNTL | (SEQ ID N^{o}54) |

Sanger 3.160 316 pos. 1201-1225 Pfa3D7|pfa1_chr4|PFD0545w|Annotation|Sanger (protein coding) hypotheti-
cal protein Location

| | |
|---|---|
| LNHLTNNLSHHNNHMNHHTSNLNHI | (SEQ ID N^{o}55) |

Sanger 3.760 376 pos. 779-816 Pfa3D7|pfa1_chr4|PFD0685c|Annotation|Sanger (protein coding) chromosome
associated protein

| | |
|---|---|
| STDINSLNDEVKKLKEELNKIRNEYDDFKNKLELLYQK | (SEQ ID N^{o}56) |

Sanger 3.970 397 pos. 1434-1458 Pfa3D7|pfa1_chr4|PFD0970c|Annotation|Sanger (protein coding) hypotheti-
cal protein Location

| | |
|---|---|
| INNMDEKINNVDEQNNNMDEKINNV | (SEQ ID N^{o}57) |

Sanger 4.590 459 pos. 2083-2116 Pfa3D7|pfa1_chr4|PFD0985w|Annotation|Sanger (protein coding) hypotheti-
cal protein Location

| | |
|---|---|
| DNNVNNMDNNVNNVDNNVNNVDNNLNNVDNNVNN | (SEQ ID N^{o}58) |

Sanger_1 3.250 325 pos. 780-813 Pfa3D7|pfa1_chr4|PFD1115c|Annotation|Sanger (protein coding) hypotheti-
cal protein Locat

| | |
|---|---|
| DVTHLTNDVTHLTNDVTHLTNDVTHLTNDVTHLT | (SEQ ID N^{o}25) |

Sanger 3.040 304 pos. 193-221 Pfa3D7|pfa1_chr5|PFE0100w|Annotation|Sanger (protein coding) hypothetical
protein Location

| | |
|---|---|
| TLIDSFNLNLSYLRESINNKKKHINKIND | (SEQ ID N^{o}59) |

Sanger 3.250 325 pos. 1012-1036 Pfa3D7|pfa1_chr5|PFE0130c|Annotation|Sanger (protein coding) hypotheti-
cal protein Location

| | |
|---|---|
| LLNLQNDIQKKNKTLEELGEEIQKY | (SEQ ID N^{o}60) |

Sanger 3.470 347 pos. 1920-1946 Pfa3D7|pfa1_chr5|PFE0570w|Annotation|Sanger (protein coding) hypotheti-
cal protein Locat

| | |
|---|---|
| NLNKVKININDLNNNIVDVNNSIHNIE | (SEQ ID N^{o}26) |

Sanger 3.140 314 pos. 1-33 Pfa3D7|pfa1_chr5|PFE0595w|Annotation|Sanger (protein coding) hypothetical
protein Locatio

| | |
|---|---|
| MSQEKISEIIKDISALKTSCEKLNSQLDELITQ | (SEQ ID N^{o}61) |

Sanger_1 2.890 289 pos. 481-505 Pfa3D7|chr6|MAL6P1.37|Annotation|Sanger (protein coding) hypothetical
protein Location

| | |
|---|---|
| YIDIKKKISELQKDNESLKIQVDRL | (SEQ ID N^{o}28) |

Sanger_2 3.250 325 pos. 845-871 Pfa3D7|chr6|MAL6P1.37|Annotation|Sanger (protein coding) hypothetical
protein Location

| | |
|---|---|
| KKRNVEEELHSLRKNYNIINEEIEEIT | (SEQ ID N^{o}27) |

Sanger 3.060 306 pos. 1399-1423 Pfa3D7|chr6|MAL6P1.44|Annotation|Sanger (protein coding) hypothetical
protein Location = co

| | |
|---|---|
| FNDVTSNVGNMNNNLTNFSNPYNSV | (SEQ ID N^{o}62) |

Sanger 3.580 358 pos. 966-1002 Pfa3D7|chr6|MAL6P1.61|Annotation|Sanger (protein coding) DNA repair
protein RAD50, putati

| | |
|---|---|
| IPLNQKVLEISKKLNNMNNNINEYKNYLSNFIHMLKE | (SEQ ID N^{o}63) |

Sanger 4.600 460 pos. 1734-1760 Pfa3D7|chr6|MAL6P1.80|Annotation|Sanger (protein coding) hypothetical
protein Location = 37

| | |
|---|---|
| NNINNINNNINNINNNINNINNNINNINNNVNNYY | (SEQ ID N^{o}64) |

Sanger 3.110 311 pos. 1436-1460 Pfa3D7|chr6|MAL6P1.307|Annotation|Sanger (protein coding) hypothetical
protein Location

| | |
|---|---|
| MNNMNNNMNNLNNSINNNNMNNINV | (SEQ ID N^{o}65) |

Sanger 3.220 322 pos. 525-549 Pfa3D7|chr6|MAL6P1.272|Annotation|Sanger (protein coding) ribonuclease,
putative Location

| | |
|---|---|
| LKKKDEEIKHLNESIYILQQNMNNL | (SEQ ID N^{o}66) |

Sanger 3.320 332 pos. 14-38 Pfa3D7|chr6|MAL6P1.254|Annotation|Sanger (protein coding) hypothetical
protein Location

| | |
|---|---|
| FDAYNEKLGSISQSIDEIKKKIDNL | (SEQ ID N^{o}67) |

Sanger 3.080 308 pos. 7581-7613 Pfa3D7|chr6|MAL6P1.147|Annotation|Sanger (protein coding) hypothetical
protein Location = c

| | |
|---|---|
| DSMNNHKDDMNNYNDNINNYVESMNNYDDIMNK | (SEQ ID N^{o}68) |

Sanger 3.570 357 pos. 3412-3441 Pfa3D7|chr6|MAL6P1.131|Annotation|Sanger (protein coding) SET-domain
protein, putative

| | |
|---|---|
| NNFVNNKMNNMNNMKNNMNNMNNIMNNIMN | (SEQ ID N^{o}69) |

Sanger_1 3.300 330 pos. 48-72 Pfa3D7|chr7|MAL7P1.13|Annotation|Sanger (protein coding) hypothetical
protein Location

| | |
|---|---|
| IDVIKNKHNKLNEKINSLEEKLIEM | (SEQ ID N^{o}70) |

Sanger_2 3.620 362 pos. 76-106 Pfa3D7|chr7|MAL7P1.13|Annotation|Sanger (protein coding) hypothetical
protein Location

| | |
|---|---|
| SNKTFEKLNEKLNDIRNDVTNYKNELEEFKN | (SEQ ID N^{o}71) |

Sanger 3.240 324 pos. 129-167 Pfa3D7|chr7|PF07_0014|Annotation|Sanger (protein coding) hypothetical
protein Location = 12

| | |
|---|---|
| NSLDYYKKVIIKLKNNINNMEEYTNNITNDINVLKAHID | (SEQ ID N^{o}72) |

Sanger 3.340 334 pos. 67-91 Pfa3D7|chr7|PF07_0021|Annotation|Sanger (protein coding) hypothetical
protein Location = 22

| | |
|---|---|
| MDTVYKNIINMSNNMTQMYNSMNNM | (SEQ ID N^{o}73) |

Sanger_1 4.670 467 pos. 785-809 Pfa3D7|chr7|PF07_0086|Annotation|Sanger (protein coding) hypothetical
protein Location

| | |
|---|---|
| VNKMNEEVNKMNEEVNKMNEEVNKM | (SEQ ID N^{o}74) |

Sanger_2 4.470 447 pos. 806-830 Pfa3D7|chr7|PF07_0086|Annotation|Sanger (protein coding) hypothetical
protein Location

| | |
|---|---|
| VNKMNKEVNKMDEEVNKMNKEVNKM | (SEQ ID N^{o}75) |

Sanger 3.580 358 pos. 122-146 Pfa3D7|chr7|PF07_0111|Annotation|Sanger (protein coding) hypothetical
protein Location = co

| | |
|---|---|
| LKSVDNYLEKINNKIKDLDDNINDR | (SEQ ID N^{o}76) |

Sanger 3.060 306 pos. 1677-1703 Pfa3D7|chr7|MAL7P1.162|Annotation|Sanger (protein coding) dynein heavy
chain, putative

| | |
|---|---|
| QMEGFQKQLDRLSDSLSKIQKALGEYL | (SEQ ID N^{o}29) |

Sanger 3.330 333 pos. 874-898 Pfa3D7|chr8|MAL8P1.12|Annotation|Sanger (protein coding) hypothetical
protein Location

| | |
|---|---|
| INNLETNINDYNKKIKEGDSQLNNI | (SEQ ID N^{o}77) |

Sanger 3.080 308 pos. 35-62 Pfa3D7|chr8|PF08_0048|Annotation|Sanger (protein coding) ATP-dependant
helicase, putative

| | |
|---|---|
| EKLKKYNNEISSLKKELDILNEKMGKCT | (SEQ ID N^{o}78) |

Sanger 3.020 302 pos. 351-375 Pfa3D7|chr8|PF08_0060|Annotation|Sanger (protein coding) asparagine-rich
antigen Location

| | |
|---|---|
| FDNVNENFESINEPYDSINEPYDNI | (SEQ ID N^{o}79) |

Sanger 3.000 300 pos. 310-334 Pfa3D7|pfa1_chr9|PF11430w|Annotation|Sanger (protein coding) hypotheti-
cal protein Locatio

| | |
|---|---|
| FNRLNKELNELRERYDHIIKHKKQM | (SEQ ID N^{o}80) |

TIGR_1 3.000 300 pos. 482-506 Pfa3D7|chr10|PF10_0195|Annotation|TIGR (protein coding) hypothetical
protein Location = com

| | |
|---|---|
| LQDLKKKKNLLQEEITNYKEEIKTL | (SEQ ID N^{o}81) |

TIGR_2 3.120 312 pos. 608-632 Pfa3D7|chr10|PF10_0195|Annotation|TIGR (protein coding) hypothetical
protein Location = com

| | |
|---|---|
| LIILDNKEKQNQQNINHLKEEINSM | (SEQ ID N^{o}82) |

TIGR 3.030 303 pos. 1755-1779 Pfa3D7|chr10|PE10_0212|Annotation|TIGR (protein coding) hypothetical
protein Location = compl

| | |
|---|---|
| INEEENSLNEMKEDINEYVEMENKL | (SEQ ID N^{o}83) |

TIGR 3.200 320 pos. 160-184 Pfa3D7|chr11|PF11_0525|Annotation|TIGR (protein coding) hypothetical
protein Location = compl

| | |
|---|---|
| MNKLKDEEKKIQEEEKKLNEELENI | (SEQ ID N^{o}84) |

TIGR 3.080 308 pos. 244-268 Pfa3D7|chr11|PF11_0108|Annotation|TIGR (protein coding) hypothetical
protein, conserved Loc

| | |
|---|---|
| LSGLQNSLSGLKTPLSGLQNSLSGL | (SEQ ID N^{o}85) |

TIGR 3.850 385 pos. 535-571 Pfa3D7|chr11|PF11_0207|Annotation|TIGR (protein coding) hypothetical
protein Location = join

| | |
|---|---|
| EEIKEEIKEVKEEIKEVKEEIKEVKEEIKEVKEEIIKE | (SEQ ID N^{o}86) |

TIGR 3.530 353 pos. 316-356 Pfa3D7|chr11|PF11_0210|Annotation|TIGR (protein coding) hypothetical
protein Location = join

| | |
|---|---|
| IEINMLTNNLLREMMKIKNKLQKLSNLLNALRSNIEKILKN | (SEQ ID N^{o}87) |

TIGR 3.020 302 pos. 145-169 Pfa3D7|chr11|PF11_0213|Annotation|TIGR (protein coding) hypothetical
protein Location = com

| | |
|---|---|
| LKLNEGLENIKQELHIIDRELKNIL | (SEQ ID N^{o}30) |

TIGR 3.560 356 pos. 3515-3550 Pfa3D7|chr11|PF11_0240|Annotation|TIGR (protein coding) dynein heavy
chain, putative Locati

| | |
|---|---|
| KGLEEANEKLQIVREKVQSLKAKLSELISQYDHAIY | (SEQ ID N^{o}88) |

TIGR 3.050 305 pos. 298-322 Pfa3D7|chr11|PF11_0262|Annotation|TIGR (protein coding) hypothetical
protein Location = 98354

| | |
|---|---|
| VETLEKKLKEIYNDEQNYKNSLQNI | (SEQ ID N^{o}89) |

TIGR_1 4.340 434 pos. 1301-1325 Pfa3D7|chr11|PF11_0317|Annotation|TIGR (protein coding) structural
maintenance of chromos

| | |
|---|---|
| IDKINENINRIKNNIKKLNDDINEL | (SEQ ID N^{o}90) |

TIGR_2 3.900 390 pos. 1372-1396 Pfa3D7|chr11|PF11_0317|Annotation|TIGR (protein coding) structural
maintenance of chromos

| | |
|---|---|
| LQKLEEDKINIYKNLNNLNQELNQL | (SEQ ID N^{o}91) |

TIGR 3.000 300 pos. 629-653 Pfa3D7|chr11|PF11_0424|Annotation|TIGR (protein coding) hypothetical
protein Location = compl

| | |
|---|---|
| LEEKTKQYNDLQNNMKTIKEQNEHL | (SEQ ID N^{o}92) |

TIGR 3.010 301 pos. 271-310 Pfa3D7|chr11|PF11_0455|Annotation|TIGR (protein coding) hypothetical
protein Location = compl

| | |
|---|---|
| RLINNIEEIYNSNCEQIQNVRDEFAELKNDLNKIMNLINI | (SEQ ID N^{o}93) |

Stanford 3.310 331 pos. 1613-1637 Pfa3D7|chr12|PFL0115w|Annotation|Stanford (protein coding) hypotheti-
cal protein Locatio

| | |
|---|---|
| IKEINKSLTEVKNELTELQKNQEEA | (SEQ ID N^{o}94) |

Stanford 3.150 315 pos. 131-161 Pfa3D7|chr12|PFL0150w|Annotation|Stanford (protein coding) origin
recognition complex I

| | |
|---|---|
| SSISSSLTNISSSLTNISSSLTNISSSLSNS | (SEQ ID N^{o}95) |

Stanford 3.280 328 pos. 263-292 Pfa3D7|chr12|PFL0250w|Annotation|Stanford (protein coding) hypothetical
protein Locatio

| | |
|---|---|
| MCELNVMENNMNNIHSNNNNISTHMDDVIE | (SEQ ID N^{o}96) |

Stanford_1 3.170 317 pos. 1463-1487 Pfa3D7|chr12|PFL0350c|Annotation|Stanford (protein coding) hypo-
thetical protein Locat

| | |
|---|---|
| LKEYSSKLQEREKKLKEKKNELQKV | (SEQ ID N^{o}97) |

Stanford_2 4.110 411 pos. 2030-2054 Pfa3D7|chr12|PFL0350c|Annotation|Stanford (protein coding) hypo-
thetical protein Locat

| | |
|---|---|
| IDNINKNINCINNDVDNINSNINNI | (SEQ ID N^{o}98) |

Stanford 3.130 313 pos. 239-272 Pfa3D7|chr12|PFL0770w|Annotation|Stanford (protein coding) seryl-tRNA
synthetase, putat

| | |
|---|---|
| KIQIEEIKKETNQINKDIDHIEMNIINLKKKIEF | (SEQ ID N^{o}99) |

Stanford 4.360 436 pos. 329-355 Pfa3D7|chr12|PFL1135c|Annotation|Stanford (protein coding) hypothetical
protein Locatio

| | |
|---|---|
| NINSVNNNINSVDNNINNVDNNINSVN | (SEQ ID N^{o}31) |

Stanford 3.220 322 pos. 297-321 Pfa3D7|chr12|PFL0125c|Annotation|Stanford (protein coding) hypothetical
protein Location

| | |
|---|---|
| IEIEREKINQLQEEIKKLQNEKNDL | (SEQ ID N^{o}100) |

Stanford 3.670 367 pos. 604-633 Pfa3D7|chr12|PFL1235c|Annotation|Stanford (protein coding) hypothetical
protein Locatio

| | |
|---|---|
| TYTLSKLNNQINELTKKINILRGNLDKARK | (SEQ ID N^{o}101) |

Stanford 3.440 344 pos. 506-540 Pfa3D7|chr12|PFL1605w|Annotation|Stanford (protein coding) hypothetical
protein Locatio

| | |
|---|---|
| KNDINVQLDDINVQLDDINVQLDDINIQLDEINLN | (SEQ ID N^{o}102) |

Stanford_1 3.380 338 pos. 190-214 Pfa3D7|chr12|PFL1930w|Annotation|Stanford (protein coding) hypotheti-
cal protein Locat

| | |
|---|---|
| KEEINEKLQLLNNDLDKKNEELNIL | (SEQ ID N^{o}103) |

Stanford_2 3.190 319 pos. 3629-3653 Pfa3D7|chr12|PFL1930w|Annotation|Stanford (protein coding) hypo-
thetical protein Loca

| | |
|---|---|
| IKNLNNEINTLNDMLKDSEEEIRML | (SEQ ID N^{o}104) |

Stanford_3 3.350 335 pos. 3963-3987 Pfa3D7|chr12|PFL1930w|Annotation|Stanford (protein coding) hypo-
thetical protein Loca

| | |
|---|---|
| LNHIINELEIKREEINQMNNKLNEL | (SEQ ID N^{o}105) |

Stanford_4 3.540 354 pos. 4776-4800 Pfa3D7|chr12|PFL1930w|Annotation|Stanford (protein coding) hypo-
thetical protein Loca

| | |
|---|---|
| VKILKKRLNSISNDLEKRTEEIEHL | (SEQ ID N^{o}106) |

Stanford_5 3.380 338 pos. 5456-5480 Pfa3D7|chr12|PFL1930w|Annotation|Stanford (protein coding) hypo-
thetical protein Loca

| | |
|---|---|
| INVLNDEITKLKNEINTYKNDLKNI | (SEQ ID N^{o}107) |

Stanford_6 3.480 348 pos. 5613-5637 Pfa3D7|chr12|PFL1930w|Annotation|Stanford (protein coding) hypo-
thetical protein Loca

| | |
|---|---|
| INKLNQQINYLQDDINSKSDNIISL | (SEQ ID N^{o}108) |

Stanford 3.260 326 pos. 117-146 Pfa3D7|chr12|PFL2310w|Annotation|Stanford (protein coding) hypothetical
protein, conser

| | |
|---|---|
| NFFLEQMENDMSSTYDKMNRINMDLSKLKR | (SEQ ID N^{o}109) |

Stanford_1 3.180 318 pos. 515-543 Pfa3D7|chr12|PFL2520w|Annotation|Stanford (protein coding) reticulo-
cyte-binding prote

| | |
|---|---|
| EKLYILEKSINKLKKLLNDINNKYQTIKK | (SEQ ID N^{o}110) |

Stanford_2 3.430 343 pos. 723-747 Pfa3D7|chr12|PFL2520w|Annotation|Stanford (protein coding) reticulo-
cyte-binding prote

| | |
|---|---|
| LNQVLEKYEELKKNINEYSKEENKL | (SEQ ID N^{o}111) |

Sanger 3.160 316 pos. 446-478 Pfa3D7|chr13_1|PF13_0065|Annotation|Sanger (protein coding) vacuolar ATP
synthase catalyt

| | |
|---|---|
| TSFSKYVRQLEQYFDNFDQDFLSLRQKISDILQ | (SEQ ID N^{o}112) |

Sanger 3.070 307 pos. 16-45 Pfa3D7|chr13_1|PF13_0088|Annotation|Sanger (protein coding) Myb1 protein
Location = complem

| | |
|---|---|
| EKLVKHLDVIDKLIENIYDNINNLNEYINK | (SEQ ID N^{o}113) |

Sanger 3.360 336 pos. 956-980 Pfa3D7|chr13_1|PF13_0097|Annotation|Sanger (protein coding) hypothetical
protein Locati

| | |
|---|---|
| INSHNNNMNNNNDNMNNMNNNNNNI | (SEQ ID N^{o}114) |

Sanger_1 3.570 357 pos. 864-888 Pfa3D7|chr13_1|MAL13P1.96|Annotation|Sanger (protein coding) chromosome
segregation pro

| | |
|---|---|
| INEIEKKIEDIEKNINITKENLKEL | (SEQ ID N^{o}115) |

Sanger_2 3.350 335 pos. 885-909 Pfa3D7|chr13_1|MAL13P1.96|Annotation|Sanger (protein coding) chromosome
segregation pro

| | |
|---|---|
| LKELENKITELQSSFSSYENEMKHV | (SEQ ID N^{o}116) |

Sanger 3.080 308 pos. 102-126 Pfa3D7|chr13_1|PF13_0107|Annotation|Sanger (protein coding) hypothetical
protein Location

| | |
|---|---|
| IDDIDRNAEQINKNYEHVNKNYEHV | (SEQ ID N^{o}117) |

Sanger 3.210 321 pos. 14-38 Pfa3D7|chr13_1|PF13_0120|Annotation|Sanger (protein coding) hypothetical
protein Location

| | |
|---|---|
| IDRQRDKINELEKKLEELRSSSEEL | (SEQ ID N^{o}118) |

Sanger 3.060 306 pos. 247-270 Pfa3D7|chr13_1|MAL13P1.147|Annotation|Sanger (protein coding) hypotheti-
cal protein Location

| | |
|---|---|
| NIIQIKNDIEQCQKSIKKIEDNLNTYE | (SEQ ID N^{o}32) |

Sanger 3.260 326 pos. 2069-2105 Pfa3D7|chr13_1|MAL13P1.176|Annotation|Sanger (protein coding) Plas-
modium falciparum ret

| | |
|---|---|
| TIVQNSYNSFSDINKNINDIDKEMKTLIPMLDELLNE | (SEQ ID N^{o}119) |

Sanger_1 3.200 320 pos. 912-936 Pfa3D7|chr13_1|PF13_0198|Annotation|Sanger (protein coding) reticulo-
cyte binding protein

| | |
|---|---|
| ISELEQEFNNNNQKLDNILQDINAM | (SEQ ID N^{o}120) |

Sanger_2 3.260 326 pos. 2139-2171 Pfa3D7|chr13_1|PF13_0198|Annotation|Sanger (protein coding) reticulo-
cyte binding protein

| | |
|---|---|
| MEIKTIVQNSYNSFSDINKNINDIDKEMKTLI | (SEQ ID N^{o}121) |

Sanger 3.380 338 pos. 618-642 Pfa3D7|chr13_1|MAL13P1.202|Annotation|Sanger (protein coding) hypotheti-
cal protein Loca

| | |
|---|---|
| MLSLQNNIDKLKKSNNNLNEDLRKK | (SEQ ID N^{o}122) |

Sanger 3.130 313 pos. 1135-1159 Pfa3D7|chr13_1|PF13_0239|Annotation|Sanger (protein coding) hypotheti-
cal protein Location

| | |
|---|---|
| MNLLREILKLMTDNIDTLKDKINEI | (SEQ ID N^{o}123) |

Sanger 3.130 313 pos. 1040-1066 Pfa3D7|chr13_1|PF13_0277|Annotation|Sanger(protein coding) hypothetical
protein Locati

| | |
|---|---|
| YIDDVDRDVENYDKGIANVDHHLNDVH | (SEQ ID N^{o}33) |

Sanger 3.400 340 pos. 284-312 Pfa3D7|chr13_1|MAL13P1.304|Annotation|Sanger (protein coding) malaria
antigen Location = jo

| | |
|---|---|
| GGLKNSNHNLNNIEMKYNTLNNNMNSINK | (SEQ ID N^{o}124) |

Sanger 4.170 417 pos. 388-414 Pfa3D7|chr13_1|MAL13P1.336|Annotation|Sanger (protein coding) hypotheti-
cal protein Locati

| | |
|---|---|
| NMNNMNNNMNNMNNNMNNNMNNMNNMN | (SEQ ID N^{o}34) |

TIGR 3.080 308 pos. 408-440 Pfa3D7|chr14|PF14_0013|Annotation|TIGR (protein coding) hypothetical
protein Location = compl

| | |
|---|---|
| PYLRRAKHNLNNLQGGINNLYSSVNVVYDNLFN | (SEQ ID N^{o}125) |

TIGR 3.410 341 pos. 397-423 Pfa3D7|chr14|PF14_0045|Annotation|TIGR (protein coding) hypothetical
protein Location = compl

| | |
|---|---|
| ARDDIQKDINKMESELINVSNEINRLD | (SEQ ID N^{o}35) |

TIGR 3.000 300 pos. 97-123 Pfa3D7|chr14|PF14_0089|Annotation|TIGR (protein coding) hypothetical protein
Location = compl

| | |
|---|---|
| NITNINKNIENIKNDMSNLNNMNDSNQ | (SEQ ID N^{o}38) |

TIGR 3.240 324 pos. 231-257 Pfa3D7|chr14|PF14_0093|Annotation|TIGR (protein coding) hypothetical
protein Location = join

| | |
|---|---|
| SSNNLSDQINILNNNIQHINSTFNNLR | (SEQ ID N^{o}36) |

TIGR 3.010 301 pos. 3419-3445 Pfa3D7|chr14|PF14_0175|Annotation|TIGR (protein coding) hypothetical
protein Location = com

| | |
|---|---|
| NNNVNNINMNNINSNVNNINNSMNNIN | (SEQ ID N^{o}37) |

TIGR 3.240 324 pos. 125-149 Pfa3D7|chr14|PF14_0385|Annotation|TIGR (protein coding) hypothetical
protein Location = join

| | |
|---|---|
| LNSLERTVASLKNNEKQLHNNIQKI | (SEQ ID N^{o}126) |

TIGR 3.370 337 pos. 7-38 Pfa3D7|chr14|PF14_0397|Annotation|TIGR (protein coding) hypothetical protein,
conserved Loc

| | |
|---|---|
| SLLDTLEKSVKGIDENIEKYNKELNVIKQKIE | (SEQ ID N^{o}127) |

TIGR 3.710 371 pos. 70-100 Pfa3D7|chr14|PF14_0444|Annotation|TIGR (protein coding) hypothetical protein
Location = compl

| | |
|---|---|
| NNEMDETINKLKKDINKLNEKIEKYDNFMKM | (SEQ ID N^{o}128) |

TIGR 3.130 313 pos. 294-318 Pfa3D7|chr14|PF14_0504|Annotation|TIGR (protein coding) hypothetical
protein Location = compl

| | |
|---|---|
| IDELENKIEKLKNELSKNSHNNNNI | (SEQ ID N^{o}129) |

TIGR_1 3.870 387 pos. 550-574 Pfa3D7|chr14|PF14_0535|Annotation|TIGR (protein coding) hypothetical
protein Location = 230

| | |
|---|---|
| INNIDDHINNIDDHINNIDDHINNI | (SEQ ID N^{o}130) |

TIGR_2 3.470 347 pos. 522-546 Pfa3D7|chr14|PF14_0535|Annotation|TIGR (protein coding) hypothetical
protein Location = 230

| | |
|---|---|
| INNIDDNKNNIDDHINNIDDHINNI | (SEQ ID N^{o}131) |

TIGR 3.070 307 pos. 166-190 Pfa3D7|chr14|PF14_0574|Annotation|TIGR (protein coding) hypothetical
protein Location = 24538

| | |
|---|---|
| LKSLNEKIKNYDSIIEEQKNQLENL | (SEQ ID N^{o}132) |
| PFB0765w\|KLEEMKQKNKELINNLNDISDELKNCIEQVNSVSRNMANVEK | (SEQ ID N^{o}134) |
| PF07_0021\|KNMDTVYKNIINMSNNMTQMYNSMNNMSHNIINASHDMMDASGNINSH | (SEQ ID N^{o}135) |
| PFB0315w\|EKMNMKMEQMDMKMEKIDVNMDQMDVKMEQMDVKMEQMDVKMKRMNK | (SEQ ID N^{o}136) |
| MAL8P1.12\|KNKLNKKWEQINDHINNLETNINDYNKKIKEGDSQLNNIQLQCENIEQKINKIKE | (SEQ ID N^{o}137) |
| PF07_0086\|NEMNKEVNKMNEEVNKMNEEVNKMNEEVNKMNKEVNKMDEEVNKMNKEVNKMNK | (SEQ ID N^{o}138) |
| MAL13P1.96\|EIINEIEKKIEDIEKNINITKENLKELENKITELQSSFSSYENEMKHVVKKIEDLEK | (SEQ ID N^{o}139) |
| PFC0345w\|QNKMENDMNIIKNDMNIMENDMNIMENDMNIIKNDMNIMEKDMNIIKNDMNIIKNNMNIIKNEMNIIKNV | (SEQ ID N^{o}140) |
| PFL0115w\|DFLDVIYYKLNIKEINKSLTEVKNELTELQKNQEEAKNILAFK | (SEQ ID N^{o}141) |
| PFL0350c\|ASIDNINKNINCINNDVDNINSNINNINDNIHKINSNVYGN | (SEQ ID N^{o}142) |
| PFL1930w\|NFIKELELQIKNLNNEINTLNDMLKDSEEEIRMLNHTLEEK | (SEQ ID N^{o}143) |
| PFL1930w\|KYKIEINVLNDEITKLKNEINTYKNDLKNINATLDFYKST | (SEQ ID N^{o}144) |
| PF13_0120\|NVLEYAELIIDRQRDKINELEKKLEELRSSSEELQKNVIK | (SEQ ID N^{o}145) |
| PF13_0239\|GIFIYNMNLLREILKLMTDNIDTLKDKINEIKCSYAFLK | (SEQ ID N^{o}146) |
| PFD0520c\|TKKLNKELSEGNKELEKLEKNIKELEETNNTLENDIKV | (SEQ ID N^{o}147) |
| PF07_0111\|NFVNNYINENILNLKSVDNYLEKINNKIKDLDDNINDR | (SEQ ID N^{o}148) |
| MAL6P1.254\|PDFDAYNEKLGSISQSIDEIKKKIDNLQKEIKVANK | (SEQ ID N^{o}149) |
| PF11_0424\|QLEEKTKQYNDLQNNMKTIKEQNEHLKNKFQSMGK | (SEQ ID N^{o}150) |
| PFD0970c\|ENINNMDEKINNVDEQNNNMDEKINNVDEKK | (SEQ ID N^{o}151) |
| PF14_0574\|EKGLKSLNEKIKNYDSIIEEQKNQLENLKM | (SEQ ID N^{o}152) |
| PF13_0198\|TISELEQEFNNNNQKLDNILQDINAMNLNINILQT | (SEQ ID N^{o}153) |
| MAL6P1.37(PEG)PFB0145c(PEG)PF14_0089\|KKRNVEEELHSLRKNYNIINEEIEEIT(PEG)TISSLSNKIVNYESKIEEL | (SEQ ID N^{o}154) |
| EKELKEVK(PEG)NITNINKNIENIKNDMSNLNNMNDSNQ | |
| PFA0170c(PEG)MAL6P1.37(PEG)PFB0145c(PEG)PF14_0089\|VNNLDSTVNYMNSTGNNINNI(PEG)KKRNVEEELHSL | (SEQ ID N^{o}155) |
| RKNYNIINEEIEEIT(PEG)TISSLSNKIVNYESKIEELEKELKEVK(PEG)NITNINKNIENIKNDMSNLNNMNDSNQ | |
| MAL13P1.304(PEG)PF08_0048\|EKLKKYNEISSLKKELDILNEKMGKCT(PEG)GGLKNSNHNLNNIEMKYNTLNNNMNSINK | (SEQ ID N^{o}156) |
| PFB0145c(PEG)MAL13P1.304(PEG)PF08_0048\|LDENEDNIKKMKSKIDDMEKEIKYR(PEG)EKLKKYNNEISSLKKELDI | (SEQ ID N^{o}157) |
| LNEKMGKCT(PEG)GGLKNSNHNLNNIEMKYNTLNNNMNSINK | |
| PFD0520c(PEG)PF08_0048(PEG)MAL6p1.37\|TKKLNKELSEGNKELEKLEKNIKELEETNNTLENDIKV(PEG)EKLKKYNN | (SEQ ID N^{o}158) |
| EISSLKKELDILNEKMGKCT(PEG)KKRNVEEELHSLRKNYNIINEEIEEIT | |
| PFD0520c\|(AcNH)KDKMHQEMEKFKKDRKNLQLNLKNTRKNHEFLKNKMQNLVLTMKKSTADDKRFQY | (SEQ ID N^{o}159) |
| PFD0520c\|EIIDKDIIYMKSRINIMRENADKNNQKYDKIVSQKDKMHQEMEK | (SEQ ID N^{o}160) |
| PFD0520c\|IKELEETNNTLENDIKVEMNK GNLYKSRLALLKKNKVRI SKAQEIIDKDIIYMK | (SEQ ID N^{o}161) |
| PFD0520c\|Fmoc-SIQRIKHLEGLTKKLNKELSEGNKELEKLEKNIKELEETNNTLENDIKVEMNKGNLYKSRLALLKKNKVRISKA | (SEQ ID N^{o}162) |
| QEIIDKDIIYMK | |
| PFD0520c\|MRHKISENEIINKIDSINLKEVKDASACMNNYTNFISIKLKKNREGIIHSIQRIKHLEGL | (SEQ ID N^{o}163) |

Among these 163 peptidic sequences, 95 coiled-coil segments were randomly selected (30-70 amino acids long with the highest coiled-coil score) and are present either in the same protein or in different ones. Most of these 95 selected antigenic peptides were recognized at various degrees (5-93%) by a panel of sera from donors living in endemic areas (Table 2).

TABLE 2
Antibody responses against 95 peptides synthesized using 37 adult sera from
Burkina Faso, 42 adult sera from Tanzania and 39 adult sera from Colombia.

| | | | Ratio | Mean | | Ratio | Mean | | Ratio | Mean |
|---|---|---|---|---|---|---|---|---|---|---|
| SEQ ID | | % | (%) | OD | % | (%) | OD | % | (%) | OD |
| N° | Proteins | Burkina Faso sera | | | Tanzanian sera | | | Colombian sera | | |
| 1 | PFA0170c | 14 | 3 | 0.168 | 38 | 10 | 0.154 | 18 | 8 | 0.151 |
| 2 | PFB0145c | 27 | 5 | 0.135 | 100 | 55 | 0.252 | 28 | 5 | 0.118 |
| 3 | | 27 | 5 | 0.146 | 48 | 7 | 0.109 | nd | nd | nd |
| 4 | | 16 | 16 | 0.254 | 40 | 12 | 0.114 | 21 | 5 | 0.118 |
| 5 | | 41 | 32 | 0.236 | 43 | 29 | 0.219 | nd | nd | nd |
| 6 | | 22 | 16 | 0.201 | 50 | 12 | 0.107 | nd | nd | nd |
| 7 | | 4 | 3 | 0.141 | 24 | 7 | 0.120 | nd | nd | nd |
| 8 | | 70 | 43 | 0.294 | 36 | 26 | 0.274 | 15 | 5 | 0.101 |
| 9 | | 32 | 8 | 0.194 | 56 | 40 | 0.258 | 26 | 10 | 0.132 |
| 10 | | 35 | 5 | 0.185 | 10 | 7 | 0.117 | nd | nd | nd |
| 11 | | 54 | 32 | 0.285 | 40 | 12 | 0.456 | 28 | 28 | 0.156 |
| 12 | | 65 | 41 | 0.242 | 74 | 45 | 0.307 | 26 | 23 | 0.174 |
| 13 | | 27 | 27 | 0.214 | 38 | 24 | 0.149 | 64 | 26 | 0.170 |
| 14 | PFC0245c | 41 | 30 | 0.266 | 40 | 21 | 0.185 | 44 | 31 | 0.215 |
| 15 | PFD0110w | 27 | 11 | 0.146 | 50 | 10 | 0.167 | nd | nd | nd |
| 16 | | 35 | 3 | 0.121 | 31 | 17 | 0.163 | nd | nd | nd |
| 17 | | 16 | 0 | 0.123 | 33 | 17 | 0.142 | nd | nd | nd |
| 18 | | 57 | 32 | 0.210 | 38 | 7 | 0.100 | nd | nd | nd |
| 19 | | 27 | 5 | 0.136 | 38 | 10 | 0.148 | nd | nd | nd |
| 20 | | 59 | 0 | 0.192 | 55 | 19 | 0.133 | nd | nd | nd |
| 21 | | 43 | 3 | 0.179 | 26 | 10 | 0.098 | nd | nd | nd |
| 22 | | 59 | 19 | 0.181 | 67 | 10 | 0.146 | nd | nd | nd |
| 23 | | 24 | 19 | 0.203 | 7 | 7 | 0.134 | nd | nd | nd |
| 24 | | 32 | 30 | 0.267 | 14 | 12 | 0.121 | nd | nd | nd |
| 25 | PFD1115c | 0 | 0 | 0.153 | 98 | 12 | 0.134 | nd | nd | nd |
| 26 | PFE0570w | 0 | 0 | 0.125 | 43 | 12 | 0.148 | 31 | 15 | 0.113 |
| 27 | MAL6P1.37 | 54 | 30 | 0.265 | 69 | 33% | 0.237 | 18 | 8 | 0.124 |
| 28 | | 22 | 3 | 0.102 | 69 | 10% | 0.113 | nd | nd | nd |
| 29 | MAL7P1.162 | 3 | 0 | 0.122 | 24 | 10% | 0.129 | nd | nd | nd |
| 30 | PF11_0213 | 16 | 5 | 0.126 | 45 | 7% | 0.128 | 23 | 3 | 0.138 |
| 31 | PFL1135c | 0 | 0 | 0.136 | 33 | 17% | 0.193 | 36 | 18 | 0.169 |
| 32 | MAL13P1.147 | 0 | 0 | 0.123 | 19 | 14% | 0.189 | nd | nd | nd |
| 33 | PF13_0277 | 19 | 8 | 0.125 | 10 | 7% | 0.121 | nd | nd | nd |
| 34 | MAL13P1.336 | 11 | 8 | 0.207 | 14 | 12% | 0.144 | nd | nd | nd |
| 35 | PF14_0045 | 43 | 24 | 0.258 | 50 | 21% | 0.180 | nd | nd | nd |
| 36 | PF14_0093 | 0 | 0 | 0.138 | 52 | 10% | 0.133 | nd | nd | nd |
| 37 | PF14_0175 | 22 | 16 | 0.358 | 21 | 17% | 0.129 | nd | nd | nd |
| 38 | PF14_0089 | 41 | 14 | 0.163 | 17 | 12% | 0.107 | 28 | 8 | 0.135 |
| 87 | PF11_0210 | 59 | 11 | 0.118 | 29 | 0% | 0.093 | nd | nd | nd |
| 45 | PFC0235w | 59 | 24 | 0.161 | 48 | 17% | 0.149 | nd | nd | nd |
| 93 | PF11_0455 | 65 | 35 | 0.188 | 40 | 0% | 0.113 | nd | nd | nd |
| 72 | PF07_0014 | 54 | 11 | 0.155 | 48 | 10% | 0.150 | nd | nd | nd |
| 56 | PFD0685c | 16 | 5 | 0.096 | 63 | 35 | 0.224 | 8 | 3 | 0.096 |
| 63 | MAL6P1.61 | 19 | 11 | 0.144 | 40 | 10 | 0.191 | nd | nd | nd |
| 86 | PF11_0207 | 70 | 57 | 0.554 | 62 | 48% | 0.263 | 59 | 36 | 0.295 |
| 119 | MAL13P1.176 | 14 | 3 | 0.097 | 20 | 13 | 0.290 | 3 | 3 | 0.098 |
| 39 | PFA0635c | 3 | 0 | 0.093 | 28 | 8 | 0.199 | 21 | 3 | 0.110 |
| 88 | PF11_0240 | 5 | 3 | 0.086 | 28 | 5 | 0.124 | nd | nd | nd |
| 64 | MAL6P1.80 | 22 | 0 | 0.108 | 15 | 0 | 0.118 | nd | nd | nd |
| 102 | PFL1605w | 43 | 32 | 0.352 | 75 | 43 | 0.220 | 21 | 8 | 0.111 |
| 58 | PFD0985w | 41 | 14 | 0.127 | 55 | 35 | 0.221 | 49 | 8 | 0.144 |
| 99 | PFL0770w | 51 | 24 | 0.180 | 36 | 24 | 0.187 | 26 | 13 | 0.108 |
| 61 | PFE0595w | 22 | 3% | 0.100 | 24% | 0% | 0.088 | nd | nd | nd |
| 68 | MAL6P1.147 | 59% | 43% | 0.288 | 76% | 36% | 0.260 | 31% | 18% | 0.199 |
| 112 | PF13_0065 | 11 | 3 | 0.119 | 35 | 5 | 0.125 | nd | nd | nd |
| 125 | PF14_0013 | 35 | 3 | 0.145 | 24 | 12 | 0.165 | nd | nd | nd |
| 121 | PF13_0198 | 27 | 11 | 0.146 | 12 | 10 | 0.161 | nd | nd | nd |
| 153 | | 35 | 22 | 0.130 | 40 | 25 | 0,143 | nd | nd | nd |
| 127 | PF14_0397 | 19 | 11 | 0.143 | 24 | 14 | 0.162 | nd | nd | nd |
| 50 | PFC0810c | 27 | 14 | 0.152 | 29 | 19 | 0.175 | nd | nd | nd |
| 71 | MAL7P1.13 | 14 | 8 | 0.121 | 14 | 5 | 0.130 | nd | nd | nd |
| 95 | PFL0150w | 5 | 0 | 0.101 | 53 | 18 | 0.156 | nd | nd | nd |
| 128 | PF14_0444 | 11 | 8 | 0.126 | 29 | 12 | 0.139 | nd | nd | nd |
| 69 | MAL6P1.131 | 22 | 14 | 0.141 | 24 | 14 | 0.178 | nd | nd | nd |
| 41 | PFB0460c | 5 | 0 | 0.123 | 21 | 0 | 0.132 | nd | nd | nd |
| 96 | PFL0250w | 51 | 32 | 0.280 | 74 | 31 | 0.281 | 56 | 28 | 0.164 |
| 101 | PFL1235c | 19 | 5 | 0.144 | 12 | 5 | 0.122 | nd | nd | nd |
| 109 | PFL2310w | 27 | 5 | 0.172 | 17 | 10 | 0.217 | nd | nd | nd |
| 113 | PF13_0088 | 22 | 14 | 0.167 | 21 | 5 | 0.122 | nd | nd | nd |
| 43 | PFB0765w | 27 | 8 | 0.161 | 17 | 7 | 0.143 | nd | nd | nd |
| 153 | PFB0765w | 57 | 32 | 0.144 | 62 | 27 | 0.154 | nd | nd | nd |
| 48 | PFC0760c | 24 | 14 | 0.188 | 21 | 5 | 0.147 | nd | nd | nd |
| 49 | | 30 | 11 | 0.127 | 67 | 45 | 0.201 | 26 | 3 | 0.103 |
| 59 | PFE0100w | 24 | 14 | 0.220 | 33 | 2 | 0.177 | nd | nd | nd |
| 110 | PFL2520w | 8 | 3 | 0.140 | 14 | 0 | 0.146 | nd | nd | nd |
| 124 | MAL13P1.304 | 57 | 27 | 0.353 | 50 | 36 | 0.259 | 51 | 10 | 0.136 |
| 78 | PF08_0048 | 54 | 43 | 0.309 | 79 | 4% | 0.359 | 51 | 36 | 0.192 |
| 135 | PF07_0021 | 41 | 14 | 0.159 | 53 | 20 | 0.143 | nd | nd | nd |
| 136 | PFB0315w | 76 | 51 | 0.176 | 93 | 65 | 0.406 | 54 | 13 | 0.109 |
| 137 | MAL8P1.12 | 89 | 57 | 0.297 | 80 | 48 | 0.246 | 33 | 5 | 0.101 |
| 138 | PF07_0086 | 89 | 51 | 0.352 | 68 | 55 | 0.378 | 15 | 21 | 0.100 |
| 139 | MAL13P1.96 | 27 | 14 | 0.144 | 70 | 48 | 0.218 | nd | nd | nd |
| 140 | PFC0345w | 51 | 46 | 0.302 | 75 | 73 | 0.505 | 15 | 5 | 0.107 |
| 141 | PFL0115w | 51 | 11 | 0.107 | 38 | 8 | 0.097 | nd | nd | nd |
| 142 | PFL0350c | 32 | 24 | 0.240 | 75 | 43 | 0.163 | nd | nd | nd |
| 143 | PFL1930w | 3 | 5 | 0.129 | 8 | 3 | 0.097 | nd | nd | nd |
| 144 | | 43 | 11 | 0.107 | 28 | 3 | 0.105 | nd | nd | nd |
| 145 | PF13_0120 | 30 | 8 | 0.099 | 35 | 8 | 0.114 | nd | nd | nd |
| 146 | PF13_0239 | 38 | 5 | 0.100 | 20 | 3 | 0.089 | nd | nd | nd |
| 147 | PFD0520c | 59 | 41 | 0.401 | 88 | 53 | 0.406 | 28 | 10 | 0.128 |
| 148 | PF07_0111 | 19 | 3 | 0.129 | 20 | 0 | 0.130 | nd | nd | nd |
| 149 | MAL6P1.254 | 41 | 5 | 0.127 | 30 | 19 | 0.198 | nd | nd | nd |
| 150 | PF11_0424 | 38 | 8 | 0.131 | 50 | 30 | 0.203 | nd | nd | nd |
| 151 | PFD0970c | 43 | 43 | 0.200 | 78 | 50 | 0.319 | 10 | 3 | 0.107 |
| 152 | PF14_0574 | 35 | 22 | 0.216 | 70 | 38 | 0.188 | nd | nd | nd |
| 133 | PF14_0089 | 62 | 59 | 0.570 | 100 | 98 | 0.825 | 85 | 74 | 0.583 |
| 159 | PFD0520c | 59 | 30 | 0.201 | 40 | 23 | 0.198 | nd | nd | nd |
| 160 | PFD0520c | 62 | 46 | 0.290 | 70 | 35 | 0.239 | nd | nd | nd |
| 161 | PFD0520c | 92 | 68 | 0.520 | 88 | 73 | 0.496 | 74 | 49 | 0.462 |
| 162 | PFD0520c | 86 | 54 | 0.447 | 88 | 78 | 0.689 | 46 | 28 | 0.354 |
| 163 | PFD0520c | 46 | 24 | 0.193 | 90 | 83 | 0.650 | 33 | 23 | 0.284 |

Results are expressed as % (value > mean negative control + 3SD), ratio (OD exp/mean OD negative control) and not determined (nd).

Thus, 71 new proteins were identified with length varying from 200 to 10,000 amino acids. Two of these proteins (protein PF14_{—}0089; PFD0520c) containing antigenic peptide (SEQ ID NO 38; 147) were chemically synthesized as a single or overlapping polypeptides and tested for recognition in ELISA assays (see Table 2). These novel protein fragments are recognized by 15-100% of adult sera from Burkina Faso, Tanzania and Colombia. Affinity purified antibodies recognize parasite infected red blood cells. In addition, the fragment derived from the protein PF14_{—}0089 has also been associated with protection in Tanzanian children (data not shown).

“Biologically active fragments” refer to a part of a sequence containing less amino acids in length than the sequence of the peptide of the invention. This sequence can be used as long as it exhibits the same properties as the native sequence from which it derives. Preferably this sequence contains less than 30%, preferably less than 60%, in particular less than 90% amino acids in length than the respective sequence of the peptide of the invention. Preferably also these sequences contain at least 20, most preferably 25, more preferably 40 and even more preferably 50 contiguous amino acids in length in common with sequence of the peptide of the invention.

These biologically active fragments can be prepared by a variety of methods and techniques known in the art such as for example chemical synthesis (e.g. multi-channel peptide synthesizer).

Furthermore, since an inherent problem with native peptides (in L-form) is the degradation by natural proteases, the peptide of the invention may be prepared in order to include D-forms and/or “retro-inverso isomers” of the peptide. Preferably, retro-inverso isomers of short parts, variants or combinations of the peptide of the invention are prepared.

By “retro-inverso isomer” is meant an isomer of a linear peptide in which the direction of the sequence is reversed and the chirality of each amino acid residue is inverted; thus, there can be no end-group complementarity.

Protecting the peptide from natural proteolysis should therefore increase the effectiveness of the specific heterobivalent or heteromultivalent compound. A higher biological activity is predicted for the retro-inverso containing peptide when compared to the non-retro-inverso containing analog owing to protection from degradation by native proteinases. Furthermore they have been shown to exhibit an increased stability and lower immunogenicity [Sela M. and Zisman E., (1997) Different roles of D-amino acids in immune phenomena—FASEB J. 11, 449].

Retro-inverso peptides are prepared for peptides of known sequence as described for example in Sela and Zisman, (1997).

Also encompassed by the present invention are modifications of the antigenic peptide (which do not normally alter primary sequence), including in vivo or in vitro chemical derivitization of peptides, e.g., acetylation or carboxylation. Also included are modifications of glycosylation, e.g., those made by modifying the glycosylation patterns of a peptide during its synthesis and processing or in further processing steps, e.g., by exposing the peptide to enzymes which affect glycosylation e.g., mammalian glycosylating or deglycosylating enzymes. Also included are sequences which have phosphorylated amino acid residues, e.g., phosphotyrosine, phosphoserine, or phosphothreonine.

Encompassed by the present invention is also a molecular chimera of the antigenic peptide of the invention. By “molecular chimera” is intended a polynucleotide or polypeptide sequence that may include a functional portion of the antigenic peptide and that will be obtained, for example, by protein chemistry techniques known by those skilled in the art.

Particular combinations of the antigenic peptide sequence or fragments or subportions thereof are also considered in the present invention. Preferably, as disclosed in example 4, the combination antigenic peptide is selected from the group comprising the peptide LR162 (SEQ ID NO 154), LR162A (SEQ ID NO 155), LR179 (SEQ ID NO 156), LR179A (SEQ ID NO 157) and LR181 (SEQ ID NO 158).

The present invention also includes variants of the antigenic peptide. The term “variants” refer to polypeptides having amino acid sequences that differ to some extent from a native sequence polypeptide, that is amino acid sequences that vary from the native 3D sequence whereby one or more amino acids are substituted by another one. The variants can occur naturally (e.g. polymorphism) or can be synthesized. Variants possess substitutions, deletions, and/or insertions at certain positions within the amino acid sequence of the native amino acid sequence. Amino acid substitutions are herein defined as exchanges within one of the following five groups:

I. Small aliphatic, nonpolar or slightly polar residues: Ala, Ser, Thr, Pro, Gly
II. Polar, positively charged residues: His, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.
Non-natural amino acids can also be introduced by chemical synthesis.

The present invention also relates to an antigenic cocktail composition of Plasmodium species that comprises at least 2 antigenic peptides of the invention or mixtures thereof. Applicants have shown that the use of an antigenic cocktail composition of the invention improves the performance of vaccines since simultaneous immune responses to antigens, which are involved in the same or in different pathogenic mechanisms, surprisingly confers greater and sustained protection. The antigenic peptides may be free or linked together. In case said antigenic peptides are linked, they are preferably linked together by the way of a linker such as PEG (Poly ethylene glycol).

Preferably, the antigenic cocktail composition is selected from the group comprising a combination with SEQ ID NO 1, 2 and 3, or SEQ ID NO 16, 33, 38 and 2 or SEQ ID NO 35, 27, 4, 34, 24, and 37.

Also encompassed by the present invention is the use of an antigenic peptide or of the antigenic cocktail composition of the invention for the preparation of a vaccine composition useful to stimulate an immune response in a mammal. “Mammal” refers to any animal classified as a mammal including humans, domestic and farm animals, and zoo, sports or pet animals, such as dogs, horses, cats, cows, monkeys, etc. Preferably the mammal is a human.

To augment the immune response elicited, it may be preferable to couple the peptides of SEQ ID NOS: 1-163, or the antigenic cocktail composition, to any carrier molecule or carrier proteins. Various protein, glycoprotein, carbohydrate or sub-unit carriers can be used, including but not limited to, tetanus toxoid/toxin, diphtheria toxoid/toxin, pseudomonas mutant carrier, bacteria outer membrane proteins, crystalline bacterial cell surface layers, various endo or exotoxins, serum albumin, gamma globulin or keyhole limpet hemocyanin, recombinant, exotoxin A, LT toxin, Cholera B toxin, Klebsiella pneumoniae OmpA, Bacterial flagella, Clostridium difficile recombinant toxin A, Peptide dendrimers (multiple antigenic peptides), pan DR epitope (PADRE), universal T-cell epitopes from tetanus toxin, Commensal bacteria, Phage (displaying peptide on and bacteria phages), attachment of peptides to recombinant IgG1 and/or other suitable constituents.

In addition, the peptides of SEQ ID NOS: 1-163, or the antigenic cocktail composition, or their conjugates with carrier proteins may be further mixed with adjuvants to elicit an immune response, as adjuvants may increase immunoprotective antibody titers or cell mediated immunity response. Such adjuvants can include, but are not limited to, MPL+TDM+CWS (SIGMA), MF59 (an oil-in-water emulsion that includes 5% squalene, 0.5% sorbitan monoleate and 0.5% sorbitan trioleate Chiron), Heat-labile toxin (HLT), CRMig (nontoxic genetic mutant of diphtheria toxin), Squalene (IDEC PHARMACEUTICALS CORP.), Ovalbumin (SIGMA), Quil A (SARGEANT, INC.), Aluminum phosphate gel (SUPERFOS BIOSECTOR), Cholera holotoxin (CT LIST BIOLOGICAL LAB.), Cholera toxin B subunit (CTB), Cholera toxin A subunit-Protein A D-fragment fusion protein, Muramyl dipeptide (MDP), Adjumera (polyphosphazene, VIRUS RESEARCH INSTITUTE), SPT (an emulsion of 5% squalene, 0.2% Tween 80, 1.25% Pluronic L121 with phosphate-buffered saline ph 7. 4), Avridine (M6 PHARMACEUTICALS), Bay R1005 (BAYER), Calcitrol (SIGMA), Calcium phosphate gel (SARGEANT INC.), CRL 1005 (Block co-polymer P1205, VAXCEL CORP.), DHEA (MERCK), DMPC (GENZYME PHARMACEUTICALS and FINE CHEMICALS). DMPG (GENZYME PHARMACEUTICALS and FINE CHEMICALS), Gamma Inulin, Gerbu Adjuvant (CC BIOTECH CORP.), GM-CSF, (IMMUNE CORP.), GMDP (PEPTECH LIMITED), Imiquimod (3M PHARMACEUTICALS), ImmTher (ENDOREX CORPORATION), ISCOM™ (ISCOTEC AB), Iscoprep 7.0.3™ (ISCOTEC AB), Loxoribine, LT-Oral Adjuvant (E. coli labile enterotoxin, protoxin, BERNA PRODUCTS CORP.), MTP-PE (CIBA-GEIGY LTD), Murametide, (VACSYN S. A.), Murapalmitine (VACSYN S. A.), Pluronic L121 (IDEC PHARMACEUTICALS CORP.), PMMA (INSTITUT FUR PHARMAZEUTISCHE TECHNOLOGIE), SAF-1 (SYNTEX ADJUVANT FORMULATION CHIRON), Stearyl tyrosine (BIOCHEM THERAPEUTIC INC.), Theramidea (IMMUNO THERAPEUTICS INC.), Threonyl-MDP (CHIRON), FREUNDS complete adjuvant, FREUNDS incomplete adjuvant, aluminum hydroxide, dimethyldioctadecyl-ammonium bromide, Adjuvax (ALPHA-BETA TECHNOLOGY), Inject Alum (PIERCE), Monophosphoryl Lipid A (RIBI IMMUNOCHEM RESEARCH), MPL+TDM (RIBI IMMUNOCHEM RESEARCH), Titermax (CYTRX), QS21, t Ribi Adjuvant System, TiterMaxGold, QS21, Adjumer, Calcitrol, CTB, LT (E. coli toxin), LPS (lipopolysaccharide), Avridine, the CpG sequences (Singh et al., 1999 Singh, M. and Hagum, D., Nature Biotechnology 1999 17: 1075-81) toxins, toxoids, glycoproteins, lipids, glycolipids, bacterial cell walls, subunits (bacterial or viral), carbohydrate moieties (mono-, di, tri-, tetra-, oligo- and polysaccharide), various liposome formulations or saponins. Combinations of various adjuvants may be used with the antigen to prepare the immunogen formulations. Adjuvants administered parentally or for the induction of mucosal immunity may also be used.

The present invention also contemplates a vaccine composition useful to stimulate an immune response in a mammal characterized in that it comprises an antigenic peptide, or an antigenic cocktail composition.

The vaccine composition can be administered by various delivery methods including intravascularly, intraperitoneally, intramuscularly, intradermally, subcutaneously, orally, nasally or by inhalation. In an embodiment, the compositions can further include a pharmaceutically acceptable excipient and/or carrier.

“Administered” or “administering”, as it applies in the present invention, refers to contact of a pharmaceutical, therapeutic, diagnostic agent or composition, to the subject, preferably a human.

The exact formulation of the vaccine composition will depend on the particular antigenic peptide, or an antigenic cocktail, or peptide-carrier conjugate, and the route of administration.

When employing more than one antigenic peptide, such two or more, or an antigenic cocktail composition, they may be used as a physical mixture or a fusion of two or more antigenic peptides to form a combination. The combination may be produced, for example, by recombinant techniques, by the use of appropriate linkers for fusing previously prepared antigenic peptides or by co-linearly synthesizing the combination with or without linkers such as, for example, PEG (poly ethylene glycol).

Also encompassed in the present invention is an antibody characterized in that it recognizes the antigenic peptide of the invention or the antigenic cocktail composition of the invention.

As used herein, an “antibody” is a protein molecule that reacts with a specific antigen and belongs to one or five distinct classes based on structural properties: IgA, IgD, IgE, IgG and IgM. The antibody can be monoclonal or polyclonal.

The antibodies of the invention can also be produced for use in passive immunotherapy, for use as diagnostic reagents, and for use as reagents in other processes such as affinity chromatography.

When used in passive immunotherapy, the antibody of the invention can be included in a pharmaceutical composition and administered to a mammal. In an embodiment, the pharmaceutical composition includes a pharmaceutically acceptable carrier, and optionally can include pharmaceutically acceptable excipients. The pharmaceutical composition can be administered intravascularly, intraperitoneally, intramuscularly, intradermally, subcutaneously, orally, nasally or by aerosol inhalation. Preferably, the pharmaceutical composition is administered intravascularly, intramuscularly, orally, nasally or by aerosol inhalation.

In an embodiment, the present invention includes an antibody, particularly a monoclonal antibodies, directed to the antigenic peptide or the antigenic cocktail composition of the invention. In particular, hybridomas can be generated using a peptide of SEQ ID NOS: 1-163 and recombinant derivative antibodies can be made using these hybridomas according to well-known genetic engineering methods (Winter G., and Milstein C., Nature, 1991, 349 293-299). Preferably, as disclosed in example 2 and Table 3, the antibody is selected from the group comprising α-8, α-9, α-11/12, α-13, α-14, α-27, α-86, α-102, α-99, α-68, α-41, α-110, α-78, α-135, α-136, α-138, α-146 and α-151 or mixtures thereof.

Other methods known in the art to humanize an antibody or produce a humanized antibody can be utilized as well. These methods can include but are not limited to the xenomouse technology developed by ABGENIX INC. (See, U.S. Pat. Nos. 6,075,181 and 6,150,584) and the methods developed by BIOVATION, BIOINVENT INTERNATIONAL AB, PROTEIN DESIGN LABS, APPLIED MOLECULAR EVOLUTION, INC., IMMGENICS PHARMACEUTICALS INC., MEDAREX INC., CAMBRIDGE ANTIBODY TECHNOLOGY, ELAN, EOS BIOTECHNOLOGY, MEDIMMUNE, MORPHOSYS, UROGENSYS INC., AVANIR PHARMACEUTICAL/XENEREX BIOSCIENCES, AFFIBODY AB, ALLEXION ANTIBODY TECHNOLOGIES, ARIUS RESEARCH INC., CELL TECH, XOMA, IDEC PHARMACEUTICALS, NEUGENESIS, EPICYTE, SEMBIOSYS GENETICS INC., BIOPROTEIN, GENZYME THERAPEUTICS, KIRIN, GEMINI SCIENCES, HEMATECH.

Likewise, other methods known in the art to screen human antibody secreting cells to SEQ ID NOS: 1-163 may be also be utilized.

As used herein, the term “humanized antibody” or other like terms means an antibody that includes a human protein sequence in at least a portion thereof. The amount of human protein sequence can vary depending on how the antibody is made.

A “fully humanized antibody” or “human antibody” as the terms or like terms are used herein can be made, for example, with xenomouse technology as discussed above or transforming human B cells with Epstein Barr virus (Traggiai et al, 2004 in Nat. Med. 10(8): 871-5). Other methods such as phage display techniques are also possible (Bradbury A R, Marks J D, 2004 in J. immunol. Methods, 290 (1-2): 29-49).

The term “recognize” refers to the fact that an antibody of the invention is directed to an antigenic peptide or an antigenic cocktail composition of the invention and binds thereto.

When recombinant techniques are employed to prepare an antigenic peptide in accordance with the present invention, nucleic acid molecules or fragments thereof encoding the polypeptides are preferably used.

Therefore the present invention also relates to a purified and isolated nucleic acid sequence comprising
i) a nucleotide sequence encoding an antigenic peptide of the invention,
ii) a nucleic acid sequence complementary to i),
iii) a degenerated nucleic acid sequence of i) or ii),
iv) a nucleic acid sequence capable of hybridizing under stringent conditions to i), ii) or iii),
v) a nucleic acid sequence encoding a truncation or an analog of an antigenic peptide of the invention,
vi) and/or a fragment of i), ii), iii), iv) or v) encoding a biologically active fragment of said antigenic peptide of the invention.

“A purified and isolated nucleic acid sequence” refers to the state in which the nucleic acid molecule encoding the antigenic peptide the invention, or nucleic acid encoding such the antigenic peptide will be, in accordance with the present invention. Nucleic acid will be free or substantially free of material with which it is naturally associated such as other polypeptides or nucleic acids with which it is found in its natural environment, or the environment in which it is prepared (e.g. cell culture) when such preparation is by recombinant nucleic acid technology practised in vitro or in vivo.

The term “nucleic acid” is intended to refer either to DNA or to RNA.

In case the nucleic acid is DNA, then DNA which can be used herein is any polydeoxynucleotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid.

DNA sequences that encode the antigenic peptide of the invention, or a fragment thereof, can be synthesized by standard chemical techniques, for example, the phosphotriester method or via automated synthesis methods and PCR methods.

The purified and isolated DNA sequence encoding the antigenic peptide according to the invention may also be produced by enzymatic techniques. Thus, restriction enzymes, which cleave nucleic acid molecules at predefined recognition sequences can be used to isolate nucleic acid sequences from larger nucleic acid molecules containing the nucleic acid sequence, such as DNA (or RNA) that codes for the antigenic peptide of the invention or for a fragment thereof.

Encompassed by the present invention is also a nucleic acid in the form of a polyribonucleotide (RNA), including, e.g., single-stranded RNA, double-stranded RNA, double-stranded RNA wherein one or both strands are composed of two or more fragments, double-stranded RNA wherein one or both strands have an uninterrupted phosphodiester backbone, RNA containing one or more single-stranded portion(s) and one or more double-stranded portion(s), double-stranded RNA wherein the RNA strands are fully complementary, double-stranded RNA wherein the RNA strands are only partially complementary, covalently crosslinked RNA, enzyme-digested RNA, sheared RNA, mRNA, chemically-synthesized RNA, semi-synthetic RNA, biosynthetic RNA, naturally-isolated RNA, labeled RNA, such as radiolabeled RNA and fluorochrome-labeled RNA, RNA containing one or more non-naturally-occurring species of nucleic acid.

The purified and isolated nucleic acid sequence, DNA or RNA, also comprises a purified and isolated nucleic acid sequence having substantial sequence identity or homology to a nucleic acid sequence encoding an antigenic peptide of the invention. Preferably, the nucleic acid will have substantial sequence identity for example at least 50%, 55%, 60%, 65%, 70%, 75%, 80%, or 85% nucleic acid identity; more preferably 90% nucleic acid identity; and most preferably at least 95%, 96%, 97%, 98%, or 99% sequence identity.

“Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Academic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988).

Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Also encompassed by the present invention is a nucleic acid sequence complementary to the antigenic peptide of the invention.

Also within the scope of the invention is a degenerated nucleic acid sequence having a sequence which differs from a nucleic acid sequence encoding the antigenic peptide of the invention, or a complementary sequence thereof, due to degeneracy in the genetic code. Such nucleic acid encodes functionally equivalent to the antigenic peptide of the invention but differs in sequence from the sequence due to degeneracy in the genetic code. This may result in silent mutations which do not affect the amino acid sequence. Any and all such nucleic acid variations are within the scope of the invention.

In addition, also considered is a nucleic acid sequence capable of hybridizing under stringent conditions, preferably high stringency conditions, to a nucleic acid sequence encoding the antigenic peptide of the invention, a nucleic acid sequence complementary thereof or a degenerated nucleic acid sequence thereof. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

The present invention also includes a purified and isolated nucleic acid encoding an antigenic peptide of the invention comprising a nucleic acid sequence encoding a truncation or an analog of the antigenic peptide.

The invention also encompasses allelic variants of the disclosed purified and isolated nucleic sequence; that is, naturally-occurring alternative forms of the isolated and purified nucleic acid that also encode antigenic peptides that are identical, homologous or related to that encoded by the purified and isolated nucleic sequences. Alternatively, non-naturally occurring variants may be produced by mutagenesis techniques or by direct synthesis.

A fragment of the disclosed purified and isolated nucleic sequence is also considered and refers to a sequence containing less nucleotides in length than the nucleic acid sequence encoding the antigenic peptide, a nucleic acid sequence complementary thereof or a degenerated nucleic acid sequence thereof. This sequence can be used as long as it exhibits the same properties as the native sequence from which it derives. Preferably this sequence contains less than 90%, preferably less than 60%, in particular less than 30% amino acids in length than the respective purified and isolated nucleic sequence of the antigenic peptide.

Yet another concern of the present invention is to provide an expression vector comprising at least one copy of the purified and isolated nucleic acid sequence encoding an antigenic peptide of the invention as described above.

The choice of an expression vector depends directly, as it is well known in the art, on the functional properties desired, e.g., antigenic peptide expression and the host cell to be transformed or transfected.

Additionally, the expression vector may further comprise a promoter operably linked to the purified and isolated nucleic acid sequence of the invention. This means that the linked isolated and purified nucleic acid sequence encoding the antigenic peptide of the present invention is under control of a suitable regulatory sequence which allows expression, i.e. transcription and translation of the inserted isolated and purified nucleic acid sequence.

As used herein, the term “promoter” designates any additional regulatory sequences as known in the art e.g a promoter and/or an enhancer, polyadenylation sites and splice junctions usually employed for the expression of the polypeptide or may include additionally one or more separate targeting sequences and may optionally encode a selectable marker. Promoters which can be used provided that such promoters are compatible with the host cell are e.g promoters obtained from the genomes of viruses such as polyoma virus, adenovirus (such as Adenovirus 2), papilloma virus (such as bovine papilloma virus), avian sarcoma virus, cytomegalovirus (such as murine or human cytomegalovirus immediate early promoter), a retrovirus, hepatitis-B virus, and Simian Virus 40 (such as SV 40 early and late promoters) or promoters obtained from heterologous mammalian promoters, such as the actin promoter or an immunoglobulin promoter or heat shock promoters.

Enhancers which can be used are e.g. enhancer sequences known from mammalian genes (globin, elastase, albumin, a-fetoprotein, and insulin) or enhancer from a eukaryotic cell virus e.g. the SV40 enhancer, the cytomegalovirus early promoter enhancer, the polyoma, and adenovirus enhancers.

A wide variety of host/expression vector combinations may be employed in expressing the nucleic acid sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Another concern of the present invention is to provide a host cell (eukaryotic or prokaryotic) comprising a purified and isolated nucleic acid sequence of the invention or an expression vector as described above.

Typically, this host cell has been transformed or transfected with a purified and isolated nucleic acid sequence of the invention or an expression vector described herein.

The term “cell transfected” or “cell transformed” or “transfected/transformed cell” means the cell into which the extracellular DNA has been introduced and thus harbours the extracellular DNA. The DNA might be introduced into the cell so that the nucleic acid is replicable either as a chromosomal integrant or as an extra chromosomal element.

Transformation or transfection of appropriate eukaryotic or prokaryotic host cells with an expression vector comprising a purified an isolated DNA sequence according to the invention is accomplished by well known methods that typically depend on the type of vector used. With regard to these methods, see e.g., Maniatis et al. 1982, Molecular Cloning, A laboratory Manual, Cold Spring Harbor Laboratory and commercially available methods.

A wide variety of unicellular host cells are useful in expressing the nucleic acid sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture. Preferably, the host cell is a bacterial cell, more preferably an E. coli cell.

The present invention is also directed to a vaccine composition for the treatment and/or prevention of malaria comprising at least the antigenic peptide of the invention, or the antigenic cocktail composition of the invention.

In a further aspect, the present invention is also directed to an acid nuclei vaccine composition for the treatment and/or prevention of malaria comprising at least a purified and isolated nucleic acid sequence of the invention, or an expression vector comprising at least one copy of the purified and isolated nucleic acid sequence of the invention, fragments thereof, molecular chimeras thereof, combinations thereof and/or variants thereof.

Yet another aspect of the present invention is a method of producing an antigenic peptide for the preparation of a vaccine composition against Plasmodium species, characterized in that it comprises the steps of:
a) identifying the presence of a sequence encoding at least one coiled coil region in the genome of said Plasmodium species,
b) selecting said sequence encoding at least one coiled coil region,
c) synthesizing an antigenic peptide corresponding to the sequence containing the at least one coiled coil region selected in step c),
d) contacting the synthesized antigenic peptide with a serum sample from at least one donor living in pathogen endemic areas; and
e) determining whether at least one antibody of said serum sample binds to the synthesized antigenic peptide.

Identification of the presence of a sequence encoding at least one coiled coil region in the genome of said Plasmodium species can be performed by a bioinformatics tool such as computer programs based on algorithms.

For example, Plasmodium falciparum 3D7 genome was used for the bioinformatics analysis (Gardner, 2002). Software named pftools and containing programs for sensitive generalized sequence profiles (Bucher et al., 1996) was used to search for the short α-helical coiled coil regions. The coiled coil profiles were constructed using an alignment of amino acid sequences corresponding to the known coiled coil domain. Two profiles containing four and five heptad repeats were used for the analysis. The coiled coil regions selected by this approach were also tested by the COILS program.

The selected α-helical coiled coil containing proteins were further tested on their possible surface location and GPI anchoring by using the following programs: identification of potential signal peptides (PSORT, http://www.psort.org/psortb/, SignalP, http://www.cbs.dtu.dk/services/SignalP/), transmembrane spanning regions (TMPRED http://www.ch.embnet.org/software/TMPRED_form.html and TMHMM http://www.cbs.dtu.dk/services/TMHMM), and GPI-anchored proteins http://mendel.imp.univie.ac.at/sat/gpi/gpi_server.html.

The same computer program, or another one, will then compare the selected sequence to the transcriptome and the proteome databases of said pathogen in order to, for example, identify proteins in the asexual erythrocytic stages (www.PlasmoDB.org). If the coiled coil score of this sequence is high and if it is present in both the transcriptome and the proteome databases, it will be selected for subsequent peptide synthesis.

Peptide synthesis is performed according to techniques well known in the art such as solid-phase Fmoc chemistry. Usually the crude peptide is then purified by RP-HPLC or any other purification techniques, and analyzed by mass spectrometry (MALDI-TOF).

Synthetic peptide is then contacted with one serum, or a panel of sera, from individuals living in malaria endemic areas, and which have been infected by the pathogen, to assess if said serum/sera bind to the synthetized antigen, and thus underlying a positive antibody response. These responses will be further analysed for the presence of IgG1 and IgG3 subclasses, which have been shown, in case the pathogen is Plasmodium falciparum, to mediate protection by the reduction of Pf growth by an antibody dependant cytotoxic inhibition (ADCI) mechanism (Oeuvray et al, 1994). Biological activity of antibodies will be tested in ADCI and invasion inhibition assays (Okoyeh et al, 1999, Haynes et al, 2002) using peptide specific antibodies purified by affinity chromatography. Usually the pathogen will be selected among the group comprising Mycobacterium tuberculosis, Influenza, Toxoplasma, Ebola virus, Streptococcus, Plasmodium falciparum, meningicoccus and staphyloccoccus

In case the pathogen is Plasmodium falciparum, the proteome and transcriptome data available in the literature (Betts, 2002, Florens et al, 2002, Bozdech et al, 2003) also allow for assessing whether these molecules are present in the erythrocytic stage.

The present invention also encompasses a diagnostic tool for determining the presence of an antigenic peptide or an antigenic cocktail composition of the invention in a sample comprising:
i) contacting the sample with an antibody directed to the antigenic peptide or the antigenic cocktail composition of the invention, and
ii) determining whether said antibody binds to a component of said sample.

The present invention further encompasses a diagnostic tool for determining the presence of antibodies directed to the antigenic peptide or to the antigenic cocktail composition of the invention in a sample comprising:
i) contacting said sample with the antigenic peptide or the antigenic cocktail composition of the invention, and
ii) determining whether antibodies bind to a component of said antigenic peptide or to said antigenic cocktail composition of the invention.

As used herein, “a sample” is an aliquot or a representative portion of a substance, material or population. For example, a sample may be a sample of blood, biological tissue, urine or feces. Preferably, the sample is blood.

As used herein, “a donor” is an individual person that lives in pathogen endemic areas and that has been, or is suspected to be, infected by said pathogen.

A “component” refers to any amino acid, nucleic acid, lipid or motif that is recognized by the antibody of the invention.

Also encompassed by the present invention is a protein that comprises at least one antigenic peptide deriving from Plasmodium species, said antigenic peptide comprising at least one coiled coil region.

Preferably the protein comprises sequences selected from the group comprising the amino acid sequences SEQ ID NO 1 to 132, SEQ ID NO 134 to SEQ ID NO 153 and SEQ ID NO 159 to SEQ ID NO 163, biologically active fragments thereof, molecular chimeras thereof, combinations thereof and/or variants thereof. Most preferably the protein is PF14_{—}0089 (containing SEQ ID NO 38) or protein PFD0520c.

Usually the protein containing at least one antigenic peptide of the invention will be chemically synthesized and tested for recognition in ELISA assays. However, any other recombinant technique or recovery method such as expressing the protein in a host cell from a nucleotide sequence encoding said protein is also considered.

The invention also encompassed a vaccine composition useful to stimulate an immune response in mammal comprising a protein containing at least one antigenic peptide of the invention and the use thereof.

Further encompassed in the present invention is an antibody that recognizes a protein containing at least one antigenic peptide of the invention and its use in diagnostic tools.

Also encompassed is a purified and isolated nucleic acid sequence comprising
i) a nucleotide sequence encoding the protein of the invention,
ii) a nucleic acid sequence complementary to i),
iii) a degenerated nucleic acid sequence of i) or ii),
iv) a nucleic acid sequence capable of hybridizing under stringent conditions to i), ii) or iii),
v) a nucleic acid sequence encoding a truncation or an analog of the protein of the invention,
vi) and/or a fragment of i), ii), iii), iv) or v).

The present invention also relates to an expression vector comprising at least one copy of said purified and isolated nucleic acid sequence encoding the protein of the invention, and a host cell comprising either said purified and isolated nucleic acid sequence or said expression vector.

Also within the scope of the present invention is a kit, said kit comprising the vaccine composition as described herein, optionally with reagents and/or instructions for use.

Alternatively, or additionally, the kit may further include other materials desirable from a commercial and user standpoint, including buffers, diluents, filters, needles, and syringes.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

### Materials

Chemicals and solvents used for the synthesis were purchased from Fluka (Buchs, Switzerland) and Novabiochem (Laufelfinger, Switzerland). Pooled human sera from Tanzania, individual adult sera from Burkina Faso and Tanzania and merozoite slides were obtained from Swiss Tropical Institute (STI, Basel, Switzerland). Sporozoite slides were obtained from Nijmegen (Holland).

### Peptide Synthesis

All the peptides were synthesized at the Department of Biochemistry (University of Lausanne, Switzerland) using solid-phase Fmoc chemistry using the Advanced ChemTech (Hatley St George, UK) AC T348 Omega multi channel synthesizer and the Applied Biosystem synthesizer 431A, Foster City, Calif.). Protein PF14_{—}0089 was synthesized from residue 47 to 186 since the 1-46 fragment was predicted to contain a leader and a trans-membrane sequence (plasmodb.org; September 2004). Crude peptides were purified by RP-HPLC (C₁₈ preparative column) and analyzed by mass spectrometry (MALDI-TOF; Applied Biosystem).

### ELISA

Microtitre 96-well plates (Maxisoip F96, Nunc, Denmark) were coated overnight (O/N) at 4° C. with 50 μl/well of peptide at a concentration of 5 μg/ml. Plates were washed using phosphate buffered saline solution containing 0.05% Tween 20 (PBS-T; Sigma, St-Louis, Mo., USA) and saturated with 5% non-fat dry milk in PBS-T for 1 hour at room temperature (RT). Serial dilutions of individual mice sera or individual human sera diluted at 1/200 in PBS-T-2.5% non-fat dry milk (PBS-T-milk) were added into the plates and incubated for 1 hour at RT. After washing, goat anti-mouse or human polyvalent immunoglobulin conjugated to alkaline phosphatase (Sigma, St-Louis, Mo., USA) diluted (1/1000) in PBS-T-milk was added and the plates incubated for 1 hour. Plates were washed and the presence of enzyme evidenced by the p-nitrophenylphosphate substrate (Sigma, St-Louis, Mo., USA). Absorbance was measured at 405 nm with a Multiskan Ascent Reader. The test sample titre was determined as the highest dilution above the mean optical density (OD) value+3 standard deviation (SD) of the negative control (normal mouse serum or human serum from naïve donors).

### Example 2

### Specific Antibody Purification by Affinity Chromatography

### Antigen-Sepharose Conjugate Preparation

5 mg of antigen was dissolved in 1 mL of coupling buffer (0.1 M NaHCO₃ containing 0.5 M NaCl, pH 8.0). The CNBr-sepharose 4B (Amersham Bioscience AB, Uppsala, Sweden) was swollen in 1 mM HCl then the gel was washed with coupling buffer. The antigen solution was added to the gel and the mixture is stirred 1 h at RT. After the coupling reaction, excess of antigen was washed away with coupling buffer. The remaining activated groups were blocked by treatment with ethanolamine (0.25 M; pH 8.0) for 30 min at RT. The gel was then washed with sodium acetate buffer (0.1 M; pH 4.0), followed by coupling buffer. The antigen-sepharose conjugate was either used or stored at 4° C. in PBS (1×) containing 1 mM azide.

### Isolation of Specific Antibody

Pooled human sera was diluted four times with PBS (1×) containing 0.5 M sodium chloride and mixed with antigen-sepharose conjugate. This mixture was then stirred gently on a wheel O/N at 4° C. After centrifugation human sera were collected and stored at −20° C. until further used. The antigen-sepharose conjugate was then washed with 5 mL of trizma base TRIS (20 mM containing 0.5 M NaCl, pH 8.0) then with 5 mL of TRIS (20 mM, pH 8.0). The elution of bound antibody was achieved with glycine (0.1 M, pH 2.5). The fractions obtained were neutralized with TRIS (1 M, pH 8).

### Indirect Fluorescence Antibody Test (IFAT)

Slides coated with Pf sporozoites were dried at RT for 30 minutes, fixed with 100% acetone at 4° C. for 10 minutes, washed 2 times in PBS-T, dried carefully and blocked with 20 μL/well of PBS 5% fetal calf serum (FCS) for 30 minutes. Slides coated with Pf merozoites were fixed with 100% acetone at −20° C. for 15 minutes and dried O/N at RT. The appropriate antibody or serum dilutions prepared in PBS 1×-2.5% non-fat dry milk, were distributed (10 μL/well) and incubated 1 h at RT (sporozoite) or 30 minutes at 37° C. (merozoite) in a humid chamber. After washings with PBS-T, goat anti-mouse polyvalent immunoglobulin conjugated with Alexa fluor 488 (Molecular Probes, Oregon, USA) diluted 1/50 in Evans blue solution (1/50000) was added (400 μL/slide) and incubated 50 minutes at RT (sporozoite) or 30 minutes at 37° C. (merozoite) in a humid chamber in the dark. After washings slides are covered with 50% glycerol, sealed and read using Zeica 200 microscope.

### Mice Immunizations

Five- to seven-weeks old female CB6F1 mice were purchased from Harlan (Horst, Holland). Mice were injected subcutaneously at the base of the tail with 20 μg of peptide dissolved in PBS (1×) or water and emulsified in Montanide ISA720. For the mix of different peptides or the fragments of Mal6P1.37 and for the fragments of PfB0145c and PfD0110w, 10 and 5 μg/mouse respectively were injected. Animals received a booster dose of antigen after 3 and 6 weeks. Ten days after each boost, mice were bled to assess the presence of specific antibody by ELISA. If positive IFAT is performed, monoclonal antibodies are isolated according to Winter and Milstein (Nature, 1991, 349 293-299). For results, see Table 7.

### Indirect Fluorescence Antibody Test (IFAT)

Slides coated with Pf sporozoites were dried at RT for 30 minutes, fixed with 100% acetone at 4° C. for 10 minutes, washed 2 times in PBS-T, dried carefully and blocked with 20 μL/well of PBS 5% fetal calf serum (FCS) for 30 minutes. Slides coated with Pf merozoites were fixed with 100% acetone at −20° C. for 15 minutes and dried O/N at RT. The appropriate antibody or serum dilutions prepared in PBS 1×-2.5% non-fat dry milk, were distributed (10 μL/well) and incubated 1 h at RT (sporozoite) or 30 minutes at 37° C. (merozoite) in a humid chamber. After washings with PBS-T, goat anti-mouse polyvalent immunoglobulin conjugated with Alexa fluor 488 (Molecular Probes, Oregon, USA) diluted 1/50 in Evans blue solution (1/50000) or anti-human IgG (Fc specific) FITC conjugate (Sigma) was added (400 μL/slide) and incubated 50 minutes at RT (sporozoite) or 30 minutes at 37° C. (merozoite) in a humid chamber in the dark. After washings slides are covered with 50% glycerol, sealed and read using Zeica 200 microscope.

### Parasites

The Uganda Palo Alto strain (FUP/C) was cultured in RPMI-1640 supplemented with 0.5% albumax I (GibcoBRL-Invitrogen). For ADCI assays, blood stages parasite cultures were synchronized by at least two successive sorbitol treatment followed, after maturation over 24 h, by floatation on 1% porcine skin gelatin type A (Sigma-Aldrich).

### Human Sera

The sera from adults from Papua New Guinea (PNG) pooled for affinity purification were collected in the Maprik district of the East Sepik Province, during a cross sectional survey in July 1992 within the framework of the Malaria vaccine Epidemiology and Evaluation Project (MVEEP) supported by the United states Agency for International Development (5). The area is highly endemic for malaria. Ethical clearance for MVEEP was obtained from the PNG Medical Research Advisory Committee. Blood was taken by venipuncture into tubes containing EDTA.

The sera were collected in the village of Goundry located in the central Mossi Plateau, Burkina Faso, between 15 and 50 km north of the capital Ouagadougou, in the province of Oubritenga. The climate is characteristic of areas of Sudanese savannah, with a dry season from November to May and a rainy season from June to October. Malaria transmission is very high during the rainy season and markedly seasonal. The ethical clearance was obtained from the Ministry of Health, Burkina Faso. After obtaining informed consent from parents and caretakers, heparine venous blood samples were collected during a cross-sectional survey during the malaria low transmission season 1998.

Sera were collected in Buenaventura the main port on the Colombian Pacific Coast after human informed consent, during a cross sectional survey carried out from February to May 2002 within the framework of a project supported by the Colombian Research Council, COLCIENCIAS. The area has unestable transmission of both P. falciparum and P. vivax malaria. Ethical clearance to draw blood from human volunteers was obtained from the Institutional Review Board of Universidad del Valle. Blood was taken by venipuncture into tubes containing EDTA and sera fractionated and stored frozen until use.

The pool of immune African globulins (PIAG) was prepared from immune individuals living in endemic areas and negative control IgG (N-IgG) was obtained from a pool of more than 1000 French adult donors with no history of malaria. Briefly, the IgG fractions from both positive and negative controls were purified using a size exclusion Trisacryl GF05M (Pall BioSepra) column followed by an ionic exchange DEAE Ceramic HyperD F column (Pall BioSepra). Purified IgG were then extensively dialyzed against RPMI and kept at 4° C. until use.

### Preparation of Human Blood Monocytes

Blood monocytes (MN) were prepared from cytapheresis samples obtained from healthy blood donors with no previous history of malaria (Lecourbe Blood Bank, Paris, France). Peripheral blood mononuclear cells (PBMC) were separated on Ficoll density gradients (J Prep, Techgen) and washed in Ca²⁺ and Mg²⁺ free HBSS buffered with 10 mM HEPES (both from GibcoBRL-Invitrogen). Cells were then distributed on polystyrene 96-well flat-bottomed culture plates (TPP, Switzerland) and adherent MN were selected by incubation for 2 h at 37° C., in a humidified 5% CO₂ atmosphere. More than 90% of the adherent cells obtained in this manner were MN as estimated by the non-specific esterase test (α-naphtyl acetate esterase; Sigma Aldrich). MN form each donor were tested prior to ADCI assays and only those without direct inhibitory effect were used in assays.

### Example 3

### Results

The screening of the Pf genome using sequence profiles identified several proteins containing putative α-helical coiled coil regions. The regions which are present in the proteome and in the transcriptome databases and have the highest coiled coil scores have been selected for subsequent peptide synthesis. Through proteome and transcriptome data available in the literature (Betts, 2002, Florens et al, 2002, Bozdech et al, 2003) Applicants have assessed if these molecules are present in the erythrocytic stage. The combined analysis/assessment identified 152 coiled-coil regions. Ninety-five coiled-coil segments, 30-70 amino acids long with the highest coiled-coil score, present either in the same protein or in different ones were selected. Most of the selected antigens were recognized at various degrees (5-93%) by a panel of sera from donors living in endemic areas (Table 2).

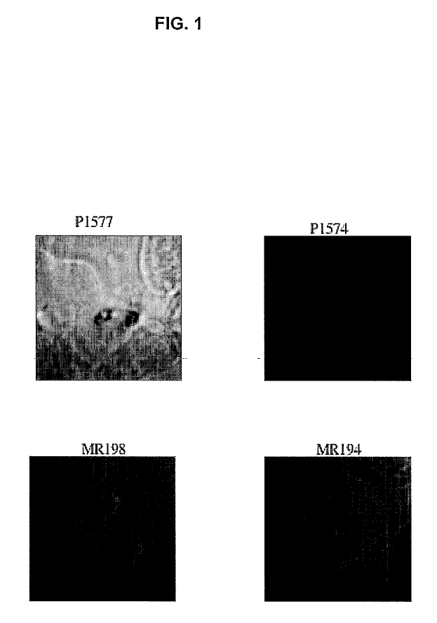

Thus, 71 new proteins were identified with length varying from 200 to 10,000 amino acids. Affinity chromatography purified antibodies specific for a number of peptides were also obtained from a pool of sera from adult donors living in Tanzania (Table 2). These antibodies are staining infected red blood cells (FIG. 1, Table 3). In addition, one of these proteins (PF14_{—}0089) was chemically synthesized and tested for recognition in ELISA assays (Table 2). This novel protein is recognized by 78-100% of adult sera from Burkina Faso and Tanzania. Affinity purified antibodies recognize parasite infected red blood cells. In addition, this protein has also been associated with protection in Tanzanian children. These Peptides are also immunogenic in mice and specific sera are positive in IFAT (Table 7). Monoclonal antibodies, which are positive in IFAT, were also isolated.

TABLE 3
The immunofluorescence antiboby test (IFAT) was done on different
stages of the parasite life cycle using affinity purified antibodies
specific for the most recognized peptides.

| | IFAT using affinity purified Abs at 10 μg/mL | | |
|---|---|---|---|
| | | | Inhibition on |
| | | | late schizonts |
| | Early/ring | Late | using the peptides |
| SEQ ID ° (proteins) | trophozoites | schizonts | at 5 mg/mL |
| 8 (PFB0145c) | + | + | + |
| 9 (PFB0145c) | + | + | − |
| 11 (PFB0145c) | + | + | +/− |
| 12 (PFB0145c) | + | + | +/− |
| 13 (PFB0145c) | + | + | +/− |
| 14 (PFC0245c) | +/− | +/− | + |
| 27 (MAL6P1.37) | + | + | +/− |
| 86 (PF11_0207) | + | + | +/− |
| 102 (PFL1605w) | + | + | +/− |
| 99 (PFL0770w) | + | + | +/− |
| 68 (MAL6P1.147) | + | + | +/− |
| 65 (PFL0250w) | + | + | +/− |
| 134 (PFC0760c) | + | + | +/− |
| 59 (MAL13P1.304) | + | + | − |
| 110 (PF08_0048) | + | + | +/− |
| 78 (PFB0315w) | + | + | +/− |
| 135 (MAL8P1.12) | + | + | − |
| 136 (PF07_0086) | + | + | − |
| 138 (PFC0345w) | + | + | + |
| 146 (PFD0520c) | + | + | + |
| 151 (PFD0970c) | + | + | +/− |

Results are expressed as weak positive (+/−), positive (+). Inhibition assays on late schizont stages was done to assess the specificity of the staining. Results are expressed as inhibition (+), no inhibition (−) and partial inhibition (+/−) observed.

### ADCI In Vitro Assay

To wells containing 2×10⁵ MN purified as described above, was added 50 μl of an asynchronous parasite culture at 0.5% parasitemia and 4% hematocrit. Wells were then supplemented with test or control Abs and the total volume adjusted to 100 μl with culture medium. After 48 h and 72 h, 50 μl of culture medium was added to each well and after 96 h the ADCI assay was stopped and the final parasitemia was determined by light microscopy on Giemsa stained smears by counting ≧50,000 red blood cells. For each antibody (Ab) tested, duplicate wells included the following controls 1) non-specific monocytic inhibition, both MN+parasite, and MN+N-IgG+parasites and 2) direct inhibition by control or test IgG, both N-IgG+parasites, and test Abs+parasites. PIAG and N-IgG were used at a final concentration of 1 mg/ml and as positive and negative controls respectively. Immunopurified test Abs were used at 15 μg/ml. The specific growth inhibitory index (SGI) which considers the parasite growth inhibition due to the effect of test Abs cooperating with MN was calculated as follows: SGI=100×[1−(% parasitemia with MN and test Abs/% parasitemia test Abs)/(% parasitemia with MN and N-IgG/% parasitemia N-IgG)].

TABLE 4
ADCI experiments, the results are expressed as
the Specific Growth Inhibitory Index (SGI).

| Affinity Purified Antibodies | SGI (%) |
|---|---|
| α-8 | 0 |
| α-9 | 60 |
| α-11/12 | Not tested |
| α-13 | 37 |
| α-14 | 46 |
| α-27 | 127 |
| α-86 | 0 |
| α-102 | Not tested |
| α-99 | 0 |
| α-68 | 0 |
| α-41 | Not tested |
| α-110 | 83 |
| α-78 | 0 |
| α-135 | 0 |
| α-136 | Not tested |
| α-138 | 0 |
| α-146 | 70 |
| α-151 | 0 |

The positive control (SGI = 100%) used is the IgG fraction purified from a pool of hyperimmune African adults sera which was previously found to confer passive protection when transferred to non-immune patients, α-8, α-9, . . . means that the antibody is directed against SEQ ID N8 or N° 9 respectively.

### Example 4

### Construction of Combination Antigenic Peptides

A combination antigenic peptides construct containing different peptide combination (Table) was co-linearly synthesized using the Fmoc chemistry and the Applied Biosystem synthesizer 431A. To avoid formation of neo-antigenic peptides, individual antigenic peptides were linked via a linker such as for example the immunological silent linker poly-ethylen-glycol (PEG 01-63-0141; NovaBiochem/Merck) as indicated in the table.

TABLE 5

| | SEQ | | |
|---|---|---|---|
| | ID | | |
| Peptide | N^{o} | Protein | Sequence |
| LR162 | 164 | MAL6P1.37(PEG)PFB0145c(PEG)PF14_0089 | KKRNVEEELHSLRKNYNIINEEIEEIT(PEG) |
| | | | TISSLSNKIVNYESKIEELEKELKEVK(PEG) |
| | | | NITNINKNIENIKNDMSNLNNMNDSNQ |
| LR162A | 155 | PFA0170c(PEG)MAL6P1.37(PEG) | VNNLDSTVNYMNSTGNNINNI(PEG) |
| | | PFB0145c(PEG)PF14_0089 | KKRNVEEELHSLRKNYNIINEEIEEIT(PEG) |
| | | | TISSLSNKIVNYESKIEELEKELKEVK(PEG) |
| | | | NITNINKNIENIKNDMSNLNNMNDSNQ |
| LR179 | 156 | MAL13P1.304(PEG)PF08_0048 | EKLKKYNNEISSLKKELDILNEKMGKCT(PEG) |
| | | | GGLKNSNHNLNNIEMKYNTLNNNMNSINK |
| LR179A | 157 | PFB0145c(PEG)MAL13P1.304(PEG)PF08_0048 | LDENEDNIKKMKSKIDDMEKEIKYR(PEG) |
| | | | EKLKKYNNEISSLKKELDILNEKMGKCT(PEG) |
| | | | GGLKNSNHNLNNIEMKYNTLNNNMNSINK |
| LR181 | 158 | PFD0520c(PEG)PF08_0048(PEG)MAL6P1.37 | TKKLNKELSEGNKELEKLEKNIKELEETNNTLENDIKV(PEG) |
| | | | EKLKKYNNEISSLKKELDILNEKMGKCT(PEG) |
| | | | KKRNVEEELHSLRKNYNIINEEIEEIT |

TABLE 6
Antibody responses against the peptides of Table 5 using 37 adult sera from
Burkina Faso, 42 adult sera from Tanzania and 39 adult sera from Colombia.

| | | Burkina Faso sera | | | Tanzania sera | | | Colombia sera | | |
|---|---|---|---|---|---|---|---|---|---|---|
| Peptide | SEQ ID | % | Ratio | Mean OD | % | Ratio | Mean OD | % | Ratio | Mean OD |
| LR162 | 154 | 81 | 49 | 0.232 | 88 | 21 | 0.169 | 54 | 49 | 0.293 |
| LR162A | 155 | 97 | 95 | 0.477 | 93 | 76 | 0.365 | 72 | 51 | 0.369 |
| LR179 | 156 | 68 | 57 | 0.323 | 73 | 48 | 0.187 | 26 | 15 | 0.125 |
| LR179A | 157 | 78 | 57 | 0.423 | 83 | 68 | 0.317 | 85 | 64 | 0.241 |
| LR181 | 158 | 73 | 68 | 0.702 | 68 | 58 | 0.391 | 15 | 10 | 0.324 |

Results are expressed as % (value > mean negative control + 3SD), ratio (OD exp/mean OD negative control) and not determined (nd).

Immunization of CBF1 Mice with One Antigenic Peptide of an Antigenic Cocktail Composition

The immunofluorescence antibody test (IFAT) was done on different stages of the parasite life cycle using affinity purified antibodies specific for the different peptides and antigenic cocktail compositions. Results are expressed as weak positive (+/−), positive (+).

TABLE 7

| Mouse immunization | | | | |
|---|---|---|---|---|
| | maximum | IFAT | | |
| SEQ ID | antibody | P. falciparum | Early/ring | Mid/late |
| N° | titres | Sporozoites | trophozoites | trophozoites |
| 1 | 218700 | − | +/− | − |
| 1 | 72900 | − | + | + |
| 2 | 100 | − | + | + |
| 3 | 900 | − | + | + |
| 35 | 24300 | − | +/− | − |
| 27 | 24300 | − | + | + |
| 38 | 218700 | − | − | +/− |
| 4 | 72900 | − | − | − |
| 34 | 100 | − | − | − |
| 16 | 100 | − | − | − |
| 33 | 900 | − | − | +/− |
| 38 | 72900 | − | − | + |
| 2 | 100 | − | − | − |
| 14 | 24300 | nd | − | − |
| 37 | 2700 | nd | − | − |
| 35 | 900 | nd | − | + |
| 27 | 24300 | nd | − | + |
| 4 | 2700 | nd | − | +/− |
| 34 | 24300 | nd | − | +/− |
| 24 | 24300 | nd | − | + |
| 37 | 900 | nd | − | +/− |
| 133 | 218700 | nd | − | + |

### Example 5

### Bio-Informatics Screening

The Plasmodium falciparum 3D7 genome was used for the bioinformatics analysis (Gardner, 2002). Software named pftools and containing programs for sensitive generalized sequence profiles (Bucher et al., 1996) was used to search for the short α-helical coiled coil regions. The coiled coil profiles were constructed using an alignment of amino acid sequences corresponding to the known coiled coil region. Two profiles containing four and five heptad repeats were used for the analysis. The coiled coil regions selected by this approach were also tested by the COILS program.

The selected α-helical coiled coil containing proteins were further tested on their possible surface location and GPI anchoring by using the following programs: identification of potential signal peptides (PSORT, http://www.psort.org/psortb/, SignalP, http://www.cbs.dtu.dk/services/SignalP/), transmembrane spanning regions (TMPRED http://www.ch.embnet.org/software/TMPRED_form.html and TMHMM http://www.cbs.dtu.dk/services/TMHMM), and GPI-anchored proteins http://mendel.imp.univie.ac.at/sat/gpi/gpi_server.html. Furthermore, the presence of the identified proteins in the asexual erythrocytic stages was also checked using the published data on the transcriptome and proteome of this stage of development of P. falciparum (www.PlasmoDB.org).

1. An antigenic peptide deriving from Plasmodium species comprising at least one coiled coil region, characterized in that said antigenic peptide is selected from the group comprising the amino acid sequences SEQ ID NO 1 to 132, SEQ ID NO 134 to SEQ ID NO 153 and SEQ ID NO 159 to SEQ ID NO 163, biologically active fragments thereof, molecular chimeras thereof, combinations thereof and/or variants thereof.

2. The antigenic peptide of claim 1, characterized in that a combination thereof is selected from the group comprising the SEQ ID NO 154, 155, 156, 157 and 158.

3. An antigenic cocktail composition deriving from Plasmodium species comprising at least 2 antigenic peptides according to claim 1.

4. The antigenic cocktail composition of claim 3 characterized in that it is selected from the group comprising a combination with
i) SEQ ID NO 1, 2 and 3, or
ii) SEQ ID NO 16, 33, 38 and 2 or
iii) SEQ ID NO 35, 27, 4, 34, 24, and 37.

5. The use of the antigenic peptide of claim 1 in the preparation of a vaccine composition for the stimulation of an immune response in a mammal.

6. A vaccine composition useful to stimulate an immune response in a mammal characterized in that it comprises the antigenic peptide of claim 1.

7. An antibody characterized in that it recognizes the antigenic peptide of claim 1.

8. The antibody of claim 7, characterized in that it is selected from the group comprising an antibody recognizing an antigenic peptide of SEQ ID NO 8, 9, 11/12, 13, 14, 27, 86, 102, 99, 68, 41, 110, 78, 135, 136, 138, 146 and 151 or mixtures thereof.

9. A purified and isolated nucleic acid sequence comprising
i) a nucleotide sequence encoding an antigenic peptide of claim 1,
ii) a nucleic acid sequence complementary to i),
iii) a degenerated nucleic acid sequence of i) or ii),
iv) a nucleic acid sequence capable of hybridizing under stringent conditions to i), ii) or iii),
v) a nucleic acid sequence encoding a truncation or an analog of the antigenic peptide
vi) and/or a fragment of i), ii), iii), iv) or v) encoding a biologically active fragment of said antigenic peptide.

10. An expression vector comprising at least one copy of the purified and isolated nucleic acid sequence of claim 9.

11. A host cell comprising a purified and isolated nucleic acid sequence of claim 9.

12. Use of the vaccine composition of claim 6, in the manufacture of a medicament for the treatment and/or prevention of malaria.

13. A method of producing an antigenic peptide for the preparation of a vaccine composition against Plasmodium species, characterized in that it comprises the steps of:
a) identifying the presence of a sequence encoding at least one coiled coil region in the genome of said Plasmodium species,
b) selecting said sequence encoding at least one coiled coil region,
c) synthesizing an antigenic peptide corresponding to the sequence containing the at least one coiled coil region selected in step b),
d) contacting the synthesized antigenic peptide with a serum sample from at least one donor living in Plasmodium species endemic areas; and
e) determining whether at least one antibody present in said serum sample binds to the synthesized antigenic peptide.

14. The method of claim 13, characterized in that step a) is performed by a bioinformatic tool.

15. A diagnostic tool for determining the presence of the antigenic peptide of claim 1 in a sample comprising:
i) contacting the sample with an antibody directed to the antigenic peptide, and
ii) determining whether said antibody binds to a component of said sample.

16. A diagnostic tool for determining the presence of antibodies to peptide of claim 1 in a sample comprising:
i) contacting said sample with the antigenic peptide of claim 1, and
ii) determining whether antibodies bind to a component of said antigenic peptide.

17. A protein characterized in that it comprises at least one antigenic peptide selected from the group comprising the amino acid sequences SEQ ID NO 2 to SEQ ID NO 37, SEQ ID NO 39 to 132, SEQ ID NO 134 to SEQ ID NO 146, SEQ ID NO 148 to SEQ ID NO 153 and SEQ ID NO 159 to SEQ ID NO 163.

18. A kit comprising the vaccine composition of claim 6, optionally with reagents and/or instructions for use.
